(19) United States
(12) Patent Application Publication
(10) Pub. No.: US 2022/0325357 A1
(43) Pub. Date: Oct. 13, 2022

(54) Method and Apparatus for Multi-Omic Simultaneous Detection of Protein Expression, Single Nucleotide Variations, and Copy Number Variations in the Same Single Cells

(71) Applicant: Mission Bio, Inc., South San Francisco, CA (US)

(72) Inventors: Dalia Dhingra, South San Francisco, CA (US); Aik Ooi, South San Francisco, CA (US); Pedro Mendez, South San Francisco, CA (US); David Ruff, South San Francisco, CA (US); Adam Sciambi, South San Francisco, CA (US)

(21) Appl. No.: 17/634,841

(22) PCT Filed: Aug. 12, 2020
(86) PCT No.: PCT/US2020/045949

§ 371 Date: Feb. 11, 2022

Related U.S. Application Data
(60) Provisional application No. 62/885,490, filed on Aug. 12, 2019.

(51) Int. Cl. C12Q 1/6886; G01N 33/574; C12Q 1/6804

(52) U.S. Cl. CPC C12Q 1/6886; G01N 33/57484; C12Q 1/6804; C12Q 1/6869

(57) ABSTRACT

Single-cell analysis of a population of cells reveals cellular genotypes (e.g., single nucleotide variants and copy number variations) and phenotypes (e.g., protein expression) of individual cells. In one scenario, individual cells can be classified according to their respective genotypes and phenotypes. In one scenario, genotypes and phenotypes of all cells in the population are informative for identifying subpopulations of cells, thereby revealing intra-population heterogeneity. The identification of subpopulations of cells is informative for improving the understanding of cellular biology, especially in the context of diseases such as cancer, and is further informative for the better design of diagnostics and therapies.

### CROSS REFERENCE

This application claims the benefit of and priority to U.S. Provisional Patent Application No. 62/885,490 filed Aug. 12, 2019, the entire disclosure of which is hereby incorporated by reference in its entirety for all purposes.

### BACKGROUND

Recent advancements in genomic analysis of tumors have revealed that cancer disease evolves by a reiterative process of somatic variation, clonal expansion and selection. Therefore, intra- and inter-tumor genomic heterogeneity have become a major area of investigation. While next-generation sequencing has contributed significantly to the understanding of cancer biology, the genetic heterogeneity of a tumor at the individual cellular level is masked with the average readout provided by a bulk measurement. Very high bulk sequence read depths are required to identify lower prevalence mutations. Rare events and mutation co-occurrence within and across select population of cells are obscured with such average signals. As such, there is difficulty in identifying heterogeneous cell populations in cells such as cancer cells, which renders cancer treatment regimen less than efficacious.

### SUMMARY

Described herein are embodiments for performing single-cell analysis of a plurality of cells to determine cellular genotypes and phenotypes of individual cells. In various embodiments, the cellular genotypes and phenotypes of individual cells are informative for discovering subpopulations of cells characterized by those genotypes and phenotypes that may not have previously been known. This is especially useful in the context of cancer where heterogeneous cell populations are often present, but not easily interrogated or discovered. The identification of subpopulations of cells is informative for improving the understanding of disease biology, and subsequently the better design of diagnostics and therapies.

Particular embodiments disclosed herein involve determining cellular genotypes directly from cellular genomic DNA. Specifically, genomic DNA is directly barcoded, amplified, and sequenced to determine cellular genotype (e.g., SNV and CNV). Such methods involving the direct determination of cellular genotypes from genomic DNA is preferable in comparison to less direct methods. For example, less direct methods involve sequencing cDNA that has been reverse transcribed from RNA transcripts, thereby providing an indirect readout of cellular genotypes. The methods disclosed herein involving direct determination of cellular genotypes from genomic DNA includes the advantages of: 1) achieve broader understanding of cellular genotype across both coding and non-coding regions (whereas less direct methods only determine cellular genotype for coding regions), 2) avoiding reverse transcription, thereby improving accuracy in calling cell mutations such as SNVs and CNVs (e.g., avoids errors and/or processing artifacts that arise due to reverse transcription), 3) reduces costs of the single-cell workflow process that arises from the inclusion of reagents needed for reverse transcription (e.g., reverse transcriptase).

Disclosed herein is a method for analyzing a plurality of cells, the method comprising: for one or more cells of the plurality of cells: encapsulating the cell in an emulsion comprising reagents, the cell comprising at least one DNA molecule and at least one analyte-bound antibody conjugated oligonucleotide; lysing the cell within the emulsion to generate a cell lysate comprising the at least one DNA molecule and the oligonucleotide; encapsulating the cell lysate comprising the at least one DNA molecule and the oligonucleotide with a reaction mixture in a second emulsion; performing a nucleic acid amplification reaction within the second emulsion using the reaction mixture to generate amplicons, the amplicons comprising: a first amplicon derived from one of the at least one DNA molecule; and a second amplicon derived from the oligonucleotide; sequencing the first amplicon and the second amplicon; determining one or more mutations of the cell using at least the sequenced first amplicon; determining a presence or absence of an analyte using at least the second amplicon; and discovering a subpopulation of cells in the plurality of cells, the subpopulation of cells characterized by the one or more mutations and the presence or absence of the analyte.

In various embodiments, the one or more mutations comprise a single nucleotide variant (SNV) or a copy number variation (CNV). In various embodiments, the one or more mutations comprise a single nucleotide variant (SNV) and a copy number variation (CNV). In various embodiments, discovering the subpopulation of cells in the plurality of cells comprises clustering the one or more cells according to the identified SNV or CNV.

In various embodiments, the SNV or CNV is identified in a gene relevant in acute lymphoblastic leukemia, acute myeloid leukemia, chronic lymphocytic leukemia, chronic myeloid leukemia, classic Hodgkin's Lymphoma, diffuse large B-cell lymphoma, follicular lymphoma, mantle cell lymphoma, multiple myeloma, myelodysplastic syndromes, myeloid, myeloproliferative neoplasms, T-cell lymphoma, breast invasive carcinoma, colon adenocarcinoma, glioblastoma multiforme, kidney renal clear cell carcinoma, liver hepatocellular carcinoma, lung adenocarcinoma, lung squamous cell carcinoma, ovarian cancer, pancreatic adenocarcinoma, prostate adenocarcinoma, or skin cutaneous melanoma. In various embodiments, the SNV or CNV is identified in any of ABL1, GNB1, KMT2D, PLCG2, GNA13, ATM, BRAF, JAK3, ADO, DNMT3A, SERPINA1, XPO1, PIM1, CCND1, FLT3, STAT3, AKT1, FAT1, CTCF, TP53, NOTCH1, KRAS, ALK, MYB, DNM2, DDX3X, CD79A, UBR5, PTEN, APC, PAX5, RUNX1, MAP2K1, CD79B, BIRC3, KMT2C, AR, CHD4, PHF6, POT1, CALR, TET2, ORAI1, OVGP1, ZMYM3, MYC, GATA2, CARD11, TP53BP1, TBL1XR1, BTK, WHSC1, MPL, FAS, CDH1, IKZF3, LRFN2, EGR2, SOCS1, PTPN11, PLCG1, CDK4, WTIP, ZFHX4, MED12, TNFRSF14, FAM46C, CDKN2A, BCOR, SORCS1, RPS15, TNFAIP3, IRF4, CBL, CSF1R, RPL22, BTG1, STAT6, PIK3CA, GNAS, CTNNB1, ASXL2, BCL11B, EZH2, DDR2, ATRX, MYD88, ARID1A, FGFR3, RAD21, EGFR, IKZF1, SMARCA4, SETD2, JAK2, ERBB2, KLF9, ERG, CREBBP, RB1, CHEK2, ERBB3, ETV6, RPL10, BCL2, DIS3, IDH1, ERBB4, NRAS, NFKBIE, NOTCH2, ESR1, HCN4, SF3B1, STAT5B, CCND3, U2AF1, FBXW7, CNOT3, EP300, CSF3R, FGFR1, USP9X, WT1, IDH2, FGFR2, SLC25A33, SH2B3, NF1, ZFP36L2, KIT, TRAF3, SETBP1, DNAH5, NCOR1, ABL1, ASXL1, GNA11, EPOR, GNAQ, XBP1, CDKN1B, USH2A, NPM1, HNF1A, FREM2, LEF1, HRAS, OPN5, ZRSR2, TSPYL2, LMO2, JAK1, B2M, TAL1, MGA, NFKBIA, ARAF, ZEB2, KDR, IL7R, SLC5A1, MYCN, PRDM1, MAP2K2, PHIP, MET, MLH1, REL, ZNF217, NOS1, MTOR, KDM6A, SPTBN5, SUZ12, UBA2, PDGFRA, PIK3R1, GATA3, CHD2, HDAC7, SMC1A, RAF1, MDGA2, USP7, SPEN, RET, ZFR2, SMAD4, ITSN1, SMARCB1, BCORL1, SMC3, SMO, RPL5, SRC, FOXO1, STK11, EBF1, PIK3CD, KMT2A, RHOA, CXCR4, PPM1D, VHL, LRP1B, and STAG2.

In various embodiments, determining presence or absence of the analyte comprises determining an expression level of the analyte, the analyte bound by the antibody conjugated to the oligonucleotide. In various embodiments, the analyte is any of HLA-DR, CD10, CD117, CD11b, CD123, CD13, CD138, CD14, CD141, CD15, CD16, CD163, CD19, CD193 (CCR3), CD1c, CD2, CD203c, CD209, CD22, CD25, CD3, CD30, CD303, CD304, CD33, CD34, CD4, CD42b, CD45RA, CD5, CD56, CD62P (P-Selectin), CD64, CD68, CD69, CD38, CD7, CD71, CD83, CD90 (Thy1), Fc epsilon RI alpha, Siglec-8, CD235a, CD49d, CD45, CD8, CD45RO, mouse IgG1, kappa, mouse IgG2a, kappa, mouse IgG2b, kappa, CD103, CD62L, CD11c, CD44, CD27, CD81, CD319 (SLAMF7), CD269 (BCMA), CD99, CD164, KCNJ3, CXCR4 (CD184), CD109, CD53, CD74, HLA-DR, DP, DQ, HLA-A, B, C, ROR1, Annexin A1, or CD20.

In various embodiments, discovering the subpopulation of cells in the plurality of cells comprises clustering the one or more cells according to the determined presence or absence of the analyte.

In various embodiments, clustering the one or more cells according to the identified SNV or CNV or clustering the one or more cells according to the determined presence of the analyte comprises performing a dimensionality reduction analysis selected from any of principal component analysis (PCA), linear discriminant analysis (LDA), T-distributed stochastic neighbor embedding (t-SNE), or uniform manifold approximation and projection (UMAP).

In various embodiments, the disclosed method further comprises: prior to encapsulating the cell in the emulsion, exposing the cell to a plurality of antibody-conjugated oligonucleotides; and washing the cell to remove excess antibody conjugated oligonucleotides. In various embodiments, the oligonucleotides conjugated to the plurality of antibodies comprise a PCR handle, a tag sequence, and a capture sequence. In various embodiments, the plurality of cells comprise cancer cells. In various embodiments, the cancer cells are any of acute lymphoblastic leukemia, acute myeloid leukemia, chronic lymphocytic leukemia, chronic myeloid leukemia, classic Hodgkin's Lymphoma, diffuse large B-cell lymphoma, follicular lymphoma, mantle cell lymphoma, multiple myeloma, myelodysplastic syndromes, myeloid, myeloproliferative neoplasms, T-cell lymphoma, breast invasive carcinoma, colon adenocarcinoma, glioblastoma multiforme, kidney renal clear cell carcinoma, liver hepatocellular carcinoma, lung adenocarcinoma, lung squamous cell carcinoma, ovarian cancer, pancreatic adenocarcinoma, prostate adenocarcinoma, or skin cutaneous melanoma.

In various embodiments, the method further comprises encapsulating a first barcode and a second barcode in the second emulsion along with the at least one DNA molecule, the oligonucleotide, and the reaction mixture. In various embodiments, the first nucleic acid comprises the first barcode. In various embodiments, the second nucleic acid comprises the second barcode. In various embodiments, the first barcode and second barcode share a same barcode sequence. In various embodiments, the first barcode and second barcode share different barcode sequences. In various embodiments, the first barcode and second barcode are releasably attached to a bead in the second emulsion.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1A depicts an overall system environment including a single cell workflow device and a computational device for conducting single-cell analysis, in accordance with an embodiment.

FIG. 1B shows an embodiment of processing single cells to generate amplified nucleic acid molecules for sequencing, in accordance with an embodiment.

FIG. 2 shows a flow process of determining cellular genotypes and phenotypes using sequence reads derived from individual cells and analyzing the cells using the cellular genotypes and phenotypes.

FIGS. 3A-3C shows the steps of analyte release in the first emulsion, in accordance with an embodiment.

FIG. 4A illustrates the priming and barcoding of an antibody-conjugated oligonucleotide, in accordance with an embodiment.

FIG. 4B illustrates the priming and barcoding of genomic DNA, in accordance with an embodiment.

FIGS. 5 and 6 show example gene targets and protein targets analyzed using the single cell workflow, in accordance with an embodiment.

FIG. 7 depicts an example computing device for implementing system and methods described in reference to FIGS. 1-6.

FIG. 8 depicts clustering of cells according to expression of different proteins.

FIG. 9A depicts four different cell lines and SNVs that differentiate the cell lines from one another.

FIG. 9B depicts clustering of cells according to protein expression, with an additional overlay of cell genotype.

FIG. 10 depicts observed gene level copy numbers for 13 genes across 4 cell lines and the correlation of the observed gene level copy numbers to known levels in the COSMIC database.

FIG. 11 depicts clustering of cells according to CNVs with an additional overlay of cell typing by SNVs.

FIG. 12A depicts clustering and identification of different subpopulations of cells from a mixed population using one of SNV, CNV, or protein data obtained from single cells.

FIG. 12B depicts clustering and identification of different subpopulations of cells from a mixed population using at least two of SNV, CNV, and protein data obtained from single cells.

### DETAILED DESCRIPTION

Definitions

Terms used in the claims and specification are defined as set forth below unless otherwise specified.

The term “subject” or “patient” are used interchangeably and encompass an organism, human or non-human, mammal or non-mammal, male or female.

The term “sample” or “test sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art.

The term “analyte” refers to a component of a cell. Cell analytes can be informative for understanding a state, behavior, or trajectory of a cell. Therefore, performing single-cell analysis of one or more analytes of a cell using the systems and methods described herein are informative for determining a state or behavior of a cell. Examples of an analyte include a nucleic acid (e.g., RNA, DNA, cDNA), a protein, a peptide, an antibody, an antibody fragment, a polysaccharide, a sugar, a lipid, a small molecule, or combinations thereof. In particular embodiments, a single-cell analysis involves analyzing two different analytes such as protein and DNA. In particular embodiments, a single-cell analysis involves analyzing three or more different analytes of a cell, such as RNA, DNA, and protein.

The phrase “cell phenotype” refers to the cell expression of one or more proteins (e.g., cellular proteomics). In various embodiments, a cell phenotype is determined using a single-cell analysis. in various embodiments, the cell phenotype can refer to the expression of a panel of proteins (e.g., a panel of proteins involved in cancer processes). In various embodiments, the protein panel includes proteins involved in any of the following hematologic malignancies: acute lymphoblastic leukemia, acute myeloid leukemia, chronic lymphocytic leukemia, chronic myeloid leukemia, classic Hodgkin's Lymphoma, diffuse large B-cell lymphoma, follicular lymphoma, mantle cell lymphoma, multiple myeloma, myelodysplastic syndromes, myeloid disease, myeloproliferative neoplasms, or T-cell lymphoma. In various embodiments, the protein panel includes proteins involved in any of the following solid tumors: breast invasive carcinoma, colon adenocarcinoma, glioblastoma multiforme, kidney renal clear cell carcinoma, liver hepatocellular carcinoma, lung adenocarcinoma, lung squamous cell carcinoma, ovarian cancer, pancreatic adenocarcinoma, prostate adenocarcinoma, or skin cutaneous melanoma. Examples proteins in the panel can include any of HLA-DR, CD10, CD117, CD11b, CD123, CD13, CD138, CD14, CD141, CD15, CD16, CD163, CD19, CD193 (CCR3), CD1c, CD2, CD203c, CD209, CD22, CD25, CD3, CD30, CD303, CD304, CD33, CD34, CD4, CD42b, CD45RA, CD5, CD56, CD62P (P-Selectin), CD64, CD68, CD69, CD38, CD7, CD71, CD83, CD90 (Thy1), Fc epsilon RI alpha, Siglec-8, CD235a, CD49d, CD45, CD8, CD45RO, mouse IgG1, kappa, mouse IgG2a, kappa, mouse IgG2b, kappa, CD103, CD62L, CD11c, CD44, CD27, CD81, CD319 (SLAMF7), CD269 (BCMA), CD99, CD164, KCNJ3, CXCR4 (CD184), CD109, CD53, CD74, HLA-DR, DP, DQ, HLA-A, B, C, ROR1, Annexin A1, or CD20.

The phrase “cell genotype” refers to the genetic makeup of the cell and can refer to one or more genes and/or the combination of alleles (e.g., homozygous or heterozygous) of a cell. The phrase cell genotype further encompasses one or more mutations of the cell including polymorphisms, single nucleotide polymorphisms (SNPs), single nucleotide variants (SNVs)), insertions, deletions, knock-ins, knock-outs, copy number variations (CNVs), duplications, translocations, and loss of heterozygosity (LOH). In various embodiments, a cell phenotype is determined using a single-cell analysis. In various embodiments, the cell phenotype can refer to the expression of a panel of genes (e.g., a panel of genes involved in cancer processes). In various embodiments, the panel includes genes involved in any of the following hematologic malignancies: acute lymphoblastic leukemia, acute myeloid leukemia, chronic lymphocytic leukemia, chronic myeloid leukemia, classic Hodgkin's Lymphoma, diffuse large B-cell lymphoma, follicular lymphoma, mantle cell lymphoma, multiple myeloma, myelodysplastic syndromes, myeloid, myeloproliferative neoplasms, or T-cell lymphoma. In various embodiments, the panel includes genes involved in any of the following solid tumors: breast invasive carcinoma, colon adenocarcinoma, glioblastoma multiforme, kidney renal clear cell carcinoma, liver hepatocellular carcinoma, lung adenocarcinoma, lung squamous cell carcinoma, ovarian cancer, pancreatic adenocarcinoma, prostate adenocarcinoma, or skin cutaneous melanoma. For example, for acute lymphoblastic leukemia, the following genes are interrogated: ASXL1, GATA2, KIT, PTPN11, TET2, DNMT3A, IDH1, KRAS, RUNX1, TP53, EZH2, IDH2, NPM1, SF3B1, U2AF1, FLT3, JAK2, NRAS, SRSF2, or WT1.

In some embodiments, the discrete entities as described herein are droplets. The terms “emulsion,” “drop,” “droplet,” and “microdroplet” are used interchangeably herein, to refer to small, generally spherically structures, containing at least a first fluid phase, e.g., an aqueous phase (e.g., water), bounded by a second fluid phase (e.g., oil) which is immiscible with the first fluid phase. In some embodiments, droplets according to the present disclosure may contain a first fluid phase, e.g., oil, bounded by a second immiscible fluid phase, e.g. an aqueous phase fluid (e.g., water). In some embodiments, the second fluid phase will be an immiscible phase carrier fluid. Thus droplets according to the present disclosure may be provided as aqueous-in-oil emulsions or oil-in-aqueous emulsions. Droplets may be sized and/or shaped as described herein for discrete entities. For example, droplets according to the present disclosure generally range from 1 μm to 1000 μm, inclusive, in diameter. Droplets according to the present disclosure may be used to encapsulate cells, nucleic acids (e.g., DNA), enzymes, reagents, reaction mixture, and a variety of other components. The term emulsion may be used to refer to an emulsion produced in, on, or by a microfluidic device and/or flowed from or applied by a microfluidic device.

The term “antibody” encompasses monoclonal antibodies (including full length monoclonal antibodies), polyclonal antibodies, multispecific antibodies (e.g., bispecific antibodies), and antibody fragments that are antigen-binding, e.g., an antibody or an antigen-binding fragment thereof. “Antibody fragment”, and all grammatical variants thereof, as used herein are defined as a portion of an intact antibody comprising the antigen binding site or variable region of the intact antibody, wherein the portion is free of the constant heavy chain domains (i.e., CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”).

“Complementarity” refers to the ability of a nucleic acid to form hydrogen bond(s) or hybridize with another nucleic acid sequence by either traditional Watson-Crick or other non-traditional types. As used herein “hybridization,” refers to the binding, duplexing, or hybridizing of a molecule only to a particular nucleotide sequence under low, medium, or highly stringent conditions, including when that sequence is present in a complex mixture (e.g., total cellular) DNA or RNA. See e.g., Ausubel, et al., Current Protocols In Molecular Biology, John Wiley & Sons, New York, N.Y., 1993. If a nucleotide at a certain position of a polynucleotide is capable of forming a Watson-Crick pairing with a nucleotide at the same position in an anti-parallel DNA or RNA strand, then the polynucleotide and the DNA or RNA molecule are complementary to each other at that position. The polynucleotide and the DNA or RNA molecule are “substantially complementary” to each other when a sufficient number of corresponding positions in each molecule are occupied by nucleotides that can hybridize or anneal with each other in order to affect the desired process. A complementary sequence is a sequence capable of annealing under stringent conditions to provide a 3′-terminal serving as the origin of synthesis of complementary chain.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988). In addition, values for percentage identity can be obtained from amino acid and nucleotide sequence alignments generated using the default settings for the AlignX component of Vector NTI Suite 8.0 (Informax, Frederick, Md.). Preferred methods to determine identity are designed to give the largest match between the sequences tested. Methods to determine identity and similarity are codified in publicly available computer programs. Example computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215:403-410 (1990)). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm may also be used to determine identity.

The terms “amplify,” “amplifying,” “amplification reaction” and their variants, refer generally to any action or process whereby at least a portion of a nucleic acid molecule (referred to as a template nucleic acid molecule) is replicated or copied into at least one additional nucleic acid molecule. The additional nucleic acid molecule optionally includes sequence that is substantially identical or substantially complementary to at least some portion of the template nucleic acid molecule. The template nucleic acid molecule can be single-stranded or double-stranded and the additional nucleic acid molecule can independently be single-stranded or double-stranded. In some embodiments, amplification includes a template-dependent in vitro enzyme-catalyzed reaction for the production of at least one copy of at least some portion of the nucleic acid molecule or the production of at least one copy of a nucleic acid sequence that is complementary to at least some portion of the nucleic acid molecule. Amplification optionally includes linear or exponential replication of a nucleic acid molecule. In some embodiments, such amplification is performed using isothermal conditions; in other embodiments, such amplification can include thermocycling. In some embodiments, the amplification is a multiplex amplification that includes the simultaneous amplification of a plurality of target sequences in a single amplification reaction. At least some of the target sequences can be situated, on the same nucleic acid molecule or on different target nucleic acid molecules included in the single amplification reaction. In some embodiments, “amplification” includes amplification of at least some portion of DNA- and RNA-based nucleic acids alone, or in combination. The amplification reaction can include single or double-stranded nucleic acid substrates and can further include any of the amplification processes known to one of ordinary skill in the art. In some embodiments, the amplification reaction includes polymerase chain reaction (PCR). In some embodiments, the amplification reaction includes an isothermal amplification reaction such as LAMP. In the present invention, the terms “synthesis” and “amplification” of nucleic acid are used. The synthesis of nucleic acid in the present invention means the elongation or extension of nucleic acid from an oligonucleotide serving as the origin of synthesis. If not only this synthesis but also the formation of other nucleic acid and the elongation or extension reaction of this formed nucleic acid occur continuously, a series of these reactions is comprehensively called amplification. The polynucleic acid produced by the amplification technology employed is generically referred to as an “amplicon” or “amplification product.”

Any nucleic acid amplification method may be utilized, such as a PCR-based assay, e.g., quantitative PCR (qPCR), or an isothermal amplification may be used to detect the presence of certain nucleic acids, e.g., genes of interest, present in discrete entities or one or more components thereof, e.g., cells encapsulated therein. Such assays can be applied to discrete entities within a microfluidic device or a portion thereof or any other suitable location. The conditions of such amplification or PCR-based assays may include detecting nucleic acid amplification over time and may vary in one or more ways.

A number of nucleic acid polymerases can be used in the amplification reactions utilized in certain embodiments provided herein, including any enzyme that can catalyze the polymerization of nucleotides (including analogs thereof) into a nucleic acid strand. Such nucleotide polymerization can occur in a template-dependent fashion. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze such polymerization. Optionally, the polymerase can be a mutant polymerase comprising one or more mutations involving the replacement of one or more amino acids with other amino acids, the insertion or deletion of one or more amino acids from the polymerase, or the linkage of parts of two or more polymerases. Typically, the polymerase comprises one or more active sites at which nucleotide binding and/or catalysis of nucleotide polymerization can occur. Some exemplary polymerases include without limitation DNA polymerases and RNA polymerases. The term “polymerase” and its variants, as used herein, also includes fusion proteins comprising at least two portions linked to each other, where the first portion comprises a peptide that can catalyze the polymerization of nucleotides into a nucleic acid strand and is linked to a second portion that comprises a second polypeptide. In some embodiments, the second polypeptide can include a reporter enzyme or a processivity-enhancing domain. Optionally, the polymerase can possess 5′ exonuclease activity or terminal transferase activity. In some embodiments, the polymerase can be optionally reactivated, for example through the use of heat, chemicals or re-addition of new amounts of polymerase into a reaction mixture. In some embodiments, the polymerase can include a hot-start polymerase or an aptamer-based polymerase that optionally can be reactivated.

The terms “target primer” or “target-specific primer” and variations thereof refer to primers that are complementary to a binding site sequence. Target primers are generally a single stranded or double-stranded polynucleotide, typically an oligonucleotide, that includes at least one sequence that is at least partially complementary to a target nucleic acid sequence.

“Forward primer binding site” and “reverse primer binding site” refers to the regions on the template DNA and/or the amplicon to which the forward and reverse primers bind. The primers act to delimit the region of the original template polynucleotide which is exponentially amplified during amplification. In some embodiments, additional primers may bind to the region 5′ of the forward primer and/or reverse primers. Where such additional primers are used, the forward primer binding site and/or the reverse primer binding site may encompass the binding regions of these additional primers as well as the binding regions of the primers themselves. For example, in some embodiments, the method may use one or more additional primers which bind to a region that lies 5′ of the forward and/or reverse primer binding region. Such a method was disclosed, for example, in WO0028082 which discloses the use of “displacement primers” or “outer primers.”

A “barcode” nucleic acid identification sequence can be incorporated into a nucleic acid primer or linked to a primer to enable independent sequencing and identification to be associated with one another via a barcode which relates information and identification that originated from molecules that existed within the same sample. There are numerous techniques that can be used to attach barcodes to the nucleic acids within a discrete entity. For example, the target nucleic acids may or may not be first amplified and fragmented into shorter pieces. The molecules can be combined with discrete entities, e.g., droplets, containing the barcodes. The barcodes can then be attached to the molecules using, for example, splicing by overlap extension. In this approach, the initial target molecules can have “adaptor” sequences added, which are molecules of a known sequence to which primers can be synthesized. When combined with the barcodes, primers can be used that are complementary to the adaptor sequences and the barcode sequences, such that the product amplicons of both target nucleic acids and barcodes can anneal to one another and, via an extension reaction such as DNA polymerization, be extended onto one another, generating a double-stranded product including the target nucleic acids attached to the barcode sequence. Alternatively, the primers that amplify that target can themselves be barcoded so that, upon annealing and extending onto the target, the amplicon produced has the barcode sequence incorporated into it. This can be applied with a number of amplification strategies, including specific amplification with PCR or non-specific amplification with, for example, MDA. An alternative enzymatic reaction that can be used to attach barcodes to nucleic acids is ligation, including blunt or sticky end ligation. In this approach, the DNA barcodes are incubated with the nucleic acid targets and ligase enzyme, resulting in the ligation of the barcode to the targets. The ends of the nucleic acids can be modified as needed for ligation by a number of techniques, including by using adaptors introduced with ligase or fragments to enable greater control over the number of barcodes added to the end of the molecule.

The term “identical” and their variants, as used herein, when used in reference to two or more sequences, refer to the degree to which the two or more sequences (e.g., nucleotide or polypeptide sequences) are the same. In the context of two or more sequences, the percent identity or homology of the sequences or subsequences thereof indicates the percentage of all monomeric units (e.g., nucleotides or amino acids) that are the same at a given position or region of the sequence (i.e., about 70% identity, preferably 75%, 80%, 85%, 90%, 95%, 97%, 98% or 99% identity). The percent identity canbe over a specified region, when compared and aligned for maximum correspondence over a comparison window, or designated region as measured using a BLAST or BLAST 2.0 sequence comparison algorithms with default parameters described below, or by manual alignment and visual inspection. Sequences are said to be “substantially identical” when there is at least 85% identity at the amino acid level or at the nucleotide level. Preferably, the identity exists over a region that is at least about 25, 50, or 100 residues in length, or across the entire length of at least one compared sequence. A typical algorithm for determining percent sequence identity and sequence similarity are the BLAST and BLAST 2.0 algorithms, which are described in Altschul et al, Nuc. Acids Res. 25:3389-3402 (1977). Other methods include the algorithms of Smith & Waterman, Adv. Appl. Math. 2:482 (1981), and Needleman & Wunsch, J. Mol. Biol. 48:443 (1970), etc. Another indication that two nucleic acid sequences are substantially identical is that the two molecules or their complements hybridize to each other under stringent hybridization conditions.

The terms “nucleic acid,” “polynucleotides,” and “oligonucleotides” refers to biopolymers of nucleotides and, unless the context indicates otherwise, includes modified and unmodified nucleotides, and DNA and RNA, and modified nucleic acid backbones. For example, in certain embodiments, the nucleic acid is a peptide nucleic acid (PNA) or a locked nucleic acid (LNA). Typically, the methods as described herein are performed using DNA as the nucleic acid template for amplification. However, nucleic acid whose nucleotide is replaced by an artificial derivative or modified nucleic acid from natural DNA or RNA is also included in the nucleic acid of the present invention insofar as it functions as a template for synthesis of complementary chain. The nucleic acid of the present invention is generally contained in a biological sample. The biological sample includes animal, plant or microbial tissues, cells, cultures and excretions, or extracts therefrom. In certain aspects, the biological sample includes intracellular parasitic genomic DNA or RNA such as virus or mycoplasma. The nucleic acid may be derived from nucleic acid contained in said biological sample. For example, genomic DNA, or cDNA synthesized from mRNA, or nucleic acid amplified on the basis of nucleic acid derived from the biological sample, are preferably used in the described methods. Unless denoted otherwise, whenever a oligonucleotide sequence is represented, it will be understood that the nucleotides are in 5′ to 3′ order from left to right and that “A” denotes deoxyadenosine, “C” denotes deoxycytidine, “G” denotes deoxyguanosine, “T” denotes deoxythymidine, and “U′ denotes uridine. Oligonucleotides are said to have “5′ ends” and “3′ ends” because mononucleotides are typically reacted to form oligonucleotides via attachment of the 5′ phosphate or equivalent group of one nucleotide to the 3′ hydroxyl or equivalent group of its neighboring nucleotide, optionally via a phosphodiester or other suitable linkage.

A template nucleic acid is a nucleic acid serving as a template for synthesizing a complementary chain in a nucleic acid amplification technique. A complementary chain having a nucleotide sequence complementary to the template has a meaning as a chain corresponding to the template, but the relationship between the two is merely relative. That is, according to the methods described herein a chain synthesized as the complementary chain can function again as a template. That is, the complementary chain can become a template. In certain embodiments, the template is derived from a biological sample, e.g., plant, animal, virus, micro-organism, bacteria, fungus, etc. In certain embodiments, the animal is a mammal, e.g., a human patient. A template nucleic acid typically comprises one or more target nucleic acid. A target nucleic acid in exemplary embodiments may comprise any single or double-stranded nucleic acid sequence that can be amplified or synthesized according to the disclosure, including any nucleic acid sequence suspected or expected to be present in a sample.

Primers and oligonucleotides used in embodiments herein comprise nucleotides. A nucleotide comprises any compound, including without limitation any naturally occurring nucleotide or analog thereof, which can bind selectively to, or can be polymerized by, a polymerase. Typically, but not necessarily, selective binding of the nucleotide to the polymerase is followed by polymerization of the nucleotide into a nucleic acid strand by the polymerase; occasionally however the nucleotide may dissociate from the polymerase without becoming incorporated into the nucleic acid strand, an event referred to herein as a “non-productive” event. Such nucleotides include not only naturally occurring nucleotides but also any analogs, regardless of their structure, that can bind selectively to, or can be polymerized by, a polymerase. While naturally occurring nucleotides typically comprise base, sugar and phosphate moieties, the nucleotides of the present disclosure can include compounds lacking any one, some or all of such moieties. For example, the nucleotide can optionally include a chain of phosphorus atoms comprising three, four, five, six, seven, eight, nine, ten or more phosphorus atoms. In some embodiments, the phosphorus chain can be attached to any carbon of a sugar ring, such as the 5′ carbon. The phosphorus chain can be linked to the sugar with an intervening O or S. In one embodiment, one or more phosphorus atoms in the chain can be part of a phosphate group having P and O. In another embodiment, the phosphorus atoms in the chain can be linked together with intervening O, NH, S, methylene, substituted methylene, ethylene, substituted ethylene, CNH₂, C(O), C(CH₂), CH₂CH₂, or C(OH)CH₂R (where R can be a 4-pyridine or 1-imidazole). In one embodiment, the phosphorus atoms in the chain can have side groups having O, BH3, or S. In the phosphorus chain, a phosphorus atom with a side group other than O can be a substituted phosphate group. In the phosphorus chain, phosphorus atoms with an intervening atom other than O can be a substituted phosphate group. Some examples of nucleotide analogs are described in Xu, U.S. Pat. No. 7,405,281.

In some embodiments, the nucleotide comprises a label and referred to herein as a “labeled nucleotide”; the label of the labeled nucleotide is referred to herein as a “nucleotide label.” In some embodiments, the label can be in the form of a fluorescent moiety (e.g. dye), luminescent moiety, or the like attached to the terminal phosphate group, i.e., the phosphate group most distal from the sugar. Some examples of nucleotides that can be used in the disclosed methods and compositions include, but are not limited to, ribonucleotides, deoxyribonucleotides, modified ribonucleotides, modified deoxyribonucleotides, ribonucleotide polyphosphates, deoxyribonucleotide polyphosphates, modified ribonucleotide polyphosphates, modified deoxyribonucleotide polyphosphates, peptide nucleotides, modified peptide nucleotides, metallonucleosides, phosphonate nucleosides, and modified phosphate-sugar backbone nucleotides, analogs, derivatives, or variants of the foregoing compounds, and the like. In some embodiments, the nucleotide can comprise non-oxygen moieties such as, for example, thio- or borano-moieties, in place of the oxygen moiety bridging the alpha phosphate and the sugar of the nucleotide, or the alpha and beta phosphates of the nucleotide, or the beta and gamma phosphates of the nucleotide, or between any other two phosphates of the nucleotide, or any combination thereof.

“Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group can include sulfur substitutions for the various oxygens, e.g. α-thio-nucleotide 5′-triphosphates. For a review of nucleic acid chemistry, see: Shabarova, Z. and Bogdanov, A. Advanced Organic Chemistry of Nucleic Acids, VCH, New York, 1994.

Overview

Described herein are embodiments for performing single-cell analyses for a plurality of cells to determine cellular genotypes and phenotypes of individual cells. Generally, the single-cell analysis involves performing targeted DNA-seq to generate sequence reads derived from genomic DNA that are used to determine the cell genotype (e.g., cell mutations such as CNVs and/or SNVs). The single-cell analysis further involves performing sequencing of oligonucleotides that are linked to antibodies, where an antibody exhibits binding affinity for a specific analyte expressed by a cell. Thus, sequence reads derived from the antibody-conjugated oligonucleotides are used to determine the cell phenotype (e.g., expression or presence of one or more analytes of the cell). The combination of cellular genotypes and phenotypes across cells in a population (e.g., a population of heterogeneous cancer cells) is useful for discerning subpopulations of cells, a subpopulation being characterized by a combination of a genotype and a phenotype. Subpopulations of cells may represent a subpopulation that was previously unknown, or a subpopulation that is unlikely to be detected using either cell genotype or phenotype alone.

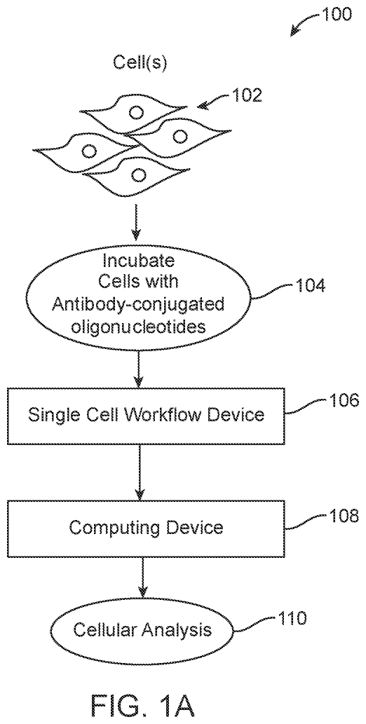

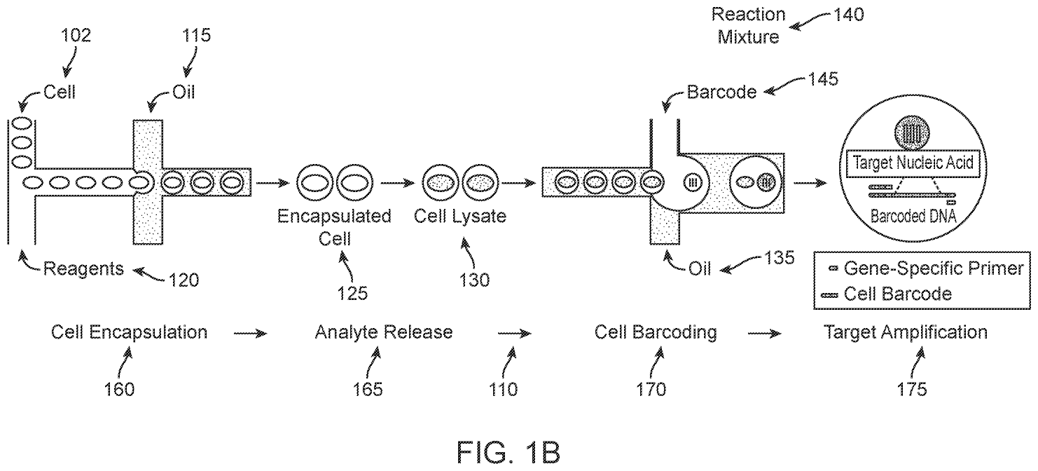

Reference is made to FIG. 1A, which depicts an overall system environment 100 including a single cell workflow device 106 and a computational device 108 for conducting single-cell analysis, in accordance with an embodiment. A population of cells 102 are obtained. In various embodiments, the cells 102 can be isolated from a test sample obtained from a subject or a patient. In various embodiments, the cells 102 are healthy cells taken from a healthy subject. In various embodiments, the cells 102 include diseased cells taken from a subject. In one embodiment, the cells 102 include cancer cells taken from a subject previously diagnosed with cancer. For example, cancer cells can be tumor cells available in the bloodstream of the subject diagnosed with cancer. As another example, cancer cells can be cells obtained through a tumor biopsy. Thus, single-cell analysis of the tumor cells enables characterization of cells of the subject's cancer. In various embodiments, the test sample is obtained from a subject following treatment of the subject (e.g., following a therapy such as cancer therapy). Thus, single-cell analysis of the cells enables characterization of cells representing the subject's response to a therapy.

At step 104, the cells 102 are incubated with antibodies. In various embodiments, an antibody exhibits binding affinity to a target analyte. For example, an antibody can exhibit binding affinity to a target epitope of a target protein.

In various embodiments, the number of cells incubated with antibodies can be 10² cells, 10³ cells, 10⁴ cells, 10⁵ cells, 10⁶ cells, or 10⁷ cells. In various embodiments, between 10³ cells and 10⁷ cells are incubated with antibodies. In various embodiments, between 10⁴ cells and 10⁶ cells are incubated with antibodies. In various embodiments, varying concentrations of antibodies are incubated with cells. In various embodiments, for an antibody in the protein panel, a concentration of 0.1 nM, 0.5 nM, 1.0 nM, 2.0 nM, 3.0 nM, 4.0 nM, 5.0 nM, 6.0 nM, 7.0 nM, 8.0 nM, 9.0 nM, 10.0 nM, 20 nM, 30 nM, 40 nM, 50 nM, 60 nM, 70 nM, 80 nM, 90 nM, or 100 nM of the antibody is incubated with cells.

In various embodiments, cells 102 are incubated with a plurality of different antibodies. In one embodiment, amongst the plurality of different antibodies, each antibody exhibits binding affinity for an analyte of a panel. For example, each antibody exhibits binding affinity for a protein of a panel. Examples of proteins included in protein panels are described herein. The incubation of cells with antibodies leads to the binding of the antibodies against target epitopes. In various embodiments, a concentration of 0.1 nM, 0.5 nM, 1.0 nM, 2.0 nM, 3.0 nM, 4.0 nM, 5.0 nM, 6.0 nM, 7.0 nM, 8.0 nM, 9.0 nM, 10.0 nM, 20 nM, 30 nM, 40 nM, 50 nM, 60 nM, 70 nM, 80 nM, 90 nM, or 100 nM for each antibody of the antibody panel is incubated with cells.

Following incubation, the cells 102 are washed (e.g., with wash buffer) to remove excess antibodies that are unbound.

In various embodiments, the antibodies are labeled with one or more oligonucleotides, also referred to as antibody oligonucleotides. Such oligonucleotides can be read out with microfluidic barcoding and DNA sequencing, thereby enabling the detection of cell analytes of interest. When an antibody binds its target, the antibody oligonucleotide is carried with it and thus allows the presence of the target analyte to be inferred based on the presence of the oligonucleotide tag. In some implementations, analyzing antibody oligonucleotides provides an estimate of the different epitopes present in the cell.

The single cell workflow device 106 refers to a device that processes individuals cells to generate nucleic acids for sequencing. In various embodiments, the single cell workflow device 106 can encapsulate individual cells into emulsions, lyse cells within the emulsions, perform cell barcoding of cell lysate in a second emulsion, and perform a nucleic amplification reaction in the second emulsion. Thus, amplified nucleic acids can be collected and sequenced. In various embodiments, the single cell workflow device 106 further includes a sequencer for sequencing the nucleic acids.

The computing device 108 is configured to receive the sequenced reads from the single cell workflow device 106. In various embodiments, the computing device 108 is communicatively coupled to the single cell workflow device 106 and therefore, directly receives the sequence reads from the single cell workflow device 106. The computing device 108 analyzes the sequence reads to generate a cellular analysis 110. In one embodiment, the computing device 108 analyzes the sequence reads to determine cellular genotypes and phenotypes. The computing device 108 uses the determined cellular genotypes and phenotypes to discover new cell subpopulations and/or to classify individual cells into cell subpopulations. Thus, in such embodiments, the cellular analysis 110 can refer to the identification of cell subpopulations or the classifications of cells into cell subpopulations.

Reference is now made to FIG. 1B, which depicts one embodiment of processing single cells to generate amplified nucleic acid molecules for sequencing. Specifically, FIG. 1B depicts a workflow process including the steps of cell encapsulation 160, analyte release 165, cell barcoding, and target amplification 175 of target nucleic acid molecules.

Generally, the cell encapsulation step 160 involves encapsulating a single cell 102 with reagents 120 into an emulsion. In various embodiments, the emulsion is formed by partitioning aqueous fluid containing the cell 102 and reagents 120 into a carrier fluid (e.g., oil 115), thereby resulting in an aqueous fluid-in-oil emulsion. The emulsion includes encapsulated cell 125 and the reagents 120. The encapsulated cell undergoes an analyte release at step 165. Generally, the reagents cause the cell to lyse, thereby generating a cell lysate 130 within the emulsion. In particular embodiments, the reagents 120 include proteases, such as proteinase K, for lysing the cell to generate a cell lysate 130. The cell lysate 130 includes the contents of the cell, which can include one or more different types of analytes (e.g., RNA transcripts, DNA, protein, lipids, or carbohydrates). In various embodiments, the different analytes of the cell lysate 130 can interact with reagents 120 within the emulsion. For example, primers in the reagents 120, such as reverse primers, can prime the analytes.

The cell barcoding step 170 involves encapsulating the cell lysate 130 into a second emulsion along with a barcode 145 and/or reaction mixture 140. In various embodiments, the second emulsion is formed by partitioning aqueous fluid containing the cell lysate 130 into immiscible oil 135. As shown in FIG. 1B, the reaction mixture 140 and barcode 145 can be introduced through a separate stream of aqueous fluid, thereby partitioning the reaction mixture 140 and barcode into the second emulsion along with the cell lysate 130.

Generally, a barcode 145 can label a target analyte to be analyzed (e.g., a target nucleic acid), which enables subsequent identification of the origin of a sequence read that is derived from the target nucleic acid. In various embodiments, multiple barcodes 145 can label multiple target nucleic acid of the cell lysate, thereby enabling the subsequent identification of the origin of large quantities of sequence reads.

Generally, the reaction mixture 140 enables the performance of a reaction, such as a nucleic acid amplification reaction. The target amplification step 175 involves amplifying target nucleic acids. For example, target nucleic acids of the cell lysate undergo amplification using the reaction mixture 140 in the second emulsion, thereby generating amplicons derived from the target nucleic acids. Although FIG. 1B depicts cell barcoding 170 and target amplification 175 as two separate steps, in various embodiments, the target nucleic acid is labeled with a barcode 145 through the nucleic acid amplification step.

As referred herein, the workflow process shown in FIG. 1B is a two-step workflow process in which analyte release 165 from the cell occurs separate from the steps of cell barcoding 170 and target amplification 175. For example, analyte release 165 from a cell occurs within a first emulsion followed by cell barcoding 170 and target amplification 175 in a second emulsion. In various embodiments, alternative workflow processes (e.g., workflow processes other than the two-step workflow process shown in FIG. 1B) can be employed. For example, the cell 102, reagents 120, reaction mixture 140, and barcode 145 can be encapsulated in an emulsion. Thus, analyte release 165 can occur within the emulsion, followed by cell barcoding 170 and target amplification 175 within the same emulsion.

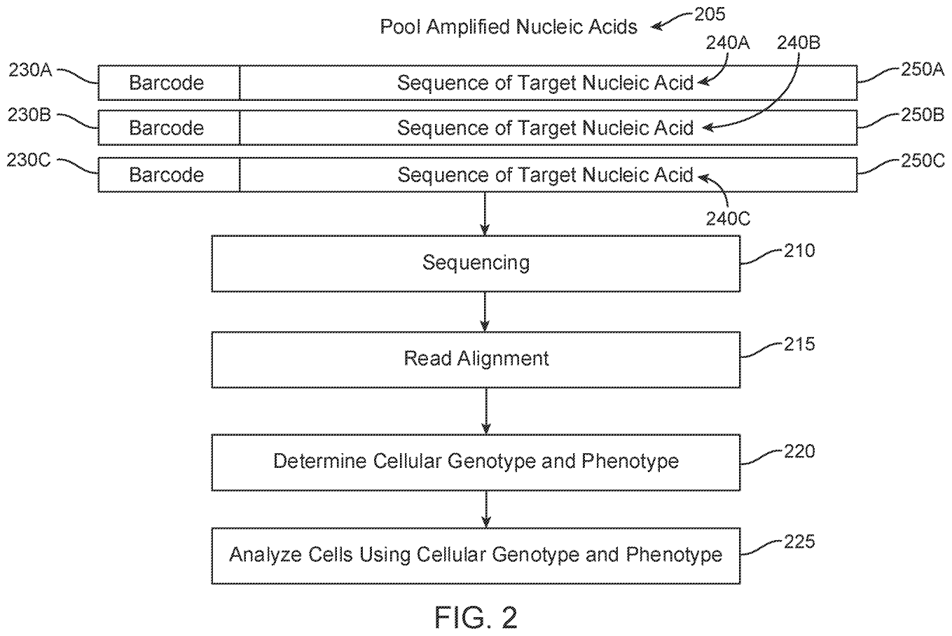

FIG. 2 is a flow process for determining cellular genotypes and phenotypes using sequence reads derived from individual cells and analyzing the cells using the cellular genotypes and phenotypes. Specifically, FIG. 2 depicts the steps of pooling amplified nucleic acids at step 205, sequencing the amplified nucleic acids, and determining a cell trajectory for a cell using the sequence reads. Generally, the flow process shown in FIG. 2 is a continuation of the workflow process shown in FIG. 1B.

For example, after target amplification at step 175 of FIG. 1B, the amplified nucleic acids 250A, 250B, and 250C are pooled at step 205 shown in FIG. 2. For example, emulsions of amplified nucleic acids are pooled and collected, and the immiscible oil of the emulsions is removed. Thus, amplified nucleic acids from multiple cells can be pooled together. FIG. 2 depicts three amplified nucleic acids 250A, 250B, and 250C but in various embodiments, pooled nucleic acids can include hundreds, thousands, or millions of nucleic acids derived from analytes of multiple cells.

In various embodiments, each amplified nucleic acid 250 includes at least a sequence of a target nucleic acid 240 and a barcode 230. In various embodiments, an amplified nucleic acid 250 can include additional sequences, such as any of a universal primer sequence (e.g., an oligo-dT sequence), a random primer sequence, a gene specific primer forward sequence, a gene specific primer reverse sequence, or one or more constant regions (e.g., PCR handles).

In various embodiments, the amplified nucleic acids 250A, 250B, and 250C are derived from the same single cell and therefore, the barcodes 230A, 230B, and 230C are the same. As such, sequencing of the barcodes 230 enables the determination that the amplified nucleic acids 250 are derived from the same cell. In various embodiments, the amplified nucleic acids 250A, 250B, and 250C are pooled and derived from different cells. Therefore, the barcodes 230A, 230B, and 230C are different from one another and sequencing of the barcodes 230 enables the determination that the amplified nucleic acids 250 are derived from different cells.

At step 210, the pooled amplified nucleic acids 250 undergo sequencing to generate sequence reads. For each amplified nucleic acid, the sequence read includes the sequence of the barcode and the target nucleic acid. Sequence reads originating from individual cells are clustered according to the barcode sequences included in the amplified nucleic acids. In various embodiments, one or more sequence reads for each single cell are aligned (e.g., to a reference genome). Aligning the sequence reads to the reference genome enables the determination of where in the genome the sequence read is derived from. For example, multiple sequence reads generated from DNA, when aligned to a position of the genome, can reveal one or more mutations present at or involving the position of the genome. In various embodiments, one or more sequence reads for each single cell do not undergo alignment. For example, sequence reads derived from antibody oligonucleotides need not be aligned to the reference genome, given that the antibody oligonucleotides are not derived from genomic DNA of the cell genome.

At step 220, aligned sequence reads for a single cell are analyzed to determine the cellular genotype and cellular phenotype of the single cell. For example, sequence reads generated from DNA transcripts are analyzed to determine one or more mutations of the cell, such as one or more CNVs and SNVs,. Sequence reads generated from antibody-conjugated oligonucleotides are used to determine the cellular phenotype, which can include the presence of absence of one or more proteins. In various embodiments, the quantity of sequence reads generated from antibody-conjugated oligonucleotides are correlated to an expression level of the one or more proteins. Taken together, the cellular genotype (e.g., one or more SNVs and CNVs) and cellular phenotype (e.g., presence/absence of proteins) provide a simultaneous view of the genomics and proteomics of a single cell.

At step 225, the cellular genotype and cellular phenotype of the cell are analyzed. In one embodiment, the cellular genotype and the cellular phenotype of the cell are used to classify the cell in a subpopulation that is characterized by the cellular genotype and phenotype. For example, a library of known cell subpopulations can be characterized based on combinations of genotypes and phenotypes. Therefore, the genotype and phenotype of the cell can be used to classify the cell in one or more cell populations that share the same or similar genotype and phenotype.

In one embodiment, the cellular genotype and cellular phenotype of the cell is used to identify cellular subpopulations. For example, the cell can be derived from a population of cells. In such embodiments, the cellular genotype and cellular phenotype of the cell is analyzed in conjunction with cellular genotypes and cellular phenotypes of other cells derived from the population of cells. In various embodiments, analyzing the cellular genotypes and cellular phenotypes of the population of cells involves performing one or both of a dimensional reduction analysis and a clustering analysis, such that cells with similar genotypes or phenotypes are localized within clusters. In various embodiments, heterogeneous subpopulations of cells can be identified from individual clusters. In various embodiments, heterogenous subpopulations of cells can be identified from even within the clusters themselves.

Identifying subpopulations of cells with differing combinations of genotypes and phenotypes can be useful for discovering subpopulations of cells in cell populations. As one example, a subpopulation of cells can refer to a cancer cell subpopulation. Thus, detection and/or identification of the presence of a cancer cell subpopulation is useful for diagnosing a subject with cancer. As another example, the population of cells may be a population of cancer cells previously thought to be homogeneous. Thus, analyzing the cellular genotypes and phenotypes of cells in the cancer cells is helpful in understanding the heterogeneity of the cancer cells, which can be used to guide the development or selection of treatments for targeting the various subpopulations of cells.

Methods for Performing Single-Cell Analysis

Encapuslation, Analyte Release, Barcoding, and Amplification

Embodiments described herein involve encapsulating one or more cells (e.g., at step 160 in FIG. 1) to perform single-cell analysis on the one or more cells. In various embodiments, encapsulating a cell with reagents is accomplished by combining an aqueous phase including the cell and reagents with an immiscible oil phase. In one embodiment, an aqueous phase including the cell and reagents are flowed together with a flowing immiscible oil phase such that water in oil emulsions are formed, where at least one emulsion includes a single cell and the reagents. In various embodiments the immiscible oil phase includes a fluorous oil, a fluorous non-ionic surfactant, or both. In various embodiments, emulsions can have an internal volume of about 0.001 to 1000 picoliters or more and can range from 0.1 to 1000 μm in diameter.

In various embodiments, the aqueous phase including the cell and reagents need not be simultaneously flowing with the immiscible oil phase. For example, the aqueous phase can be flowed to contact a stationary reservoir of the immiscible oil phase, thereby enabling the budding of water in oil emulsions within the stationary oil reservoir.

In various embodiments, combining the aqueous phase and the immiscible oil phase can be performed in a microfluidic device. For example, the aqueous phase can flow through a microchannel of the microfluidic device to contact the immiscible oil phase, which is simultaneously flowing through a separate microchannel or is held in a stationary reservoir of the microfluidic device. The encapsulated cell and reagents within an emulsion can then be flowed through the microfluidic device to undergo cell lysis.

Further example embodiments of adding reagents and cells to emulsions can include merging emulsions that separately contain the cells and reagents or picoinjecting reagents into an emulsion. Further description of example embodiments is described in U.S. application Ser. No. 14/420,646, which is hereby incorporated by reference in its entirety.

The encapsulated cell in an emulsion is lysed to generate cell lysate. In various embodiments, a cell is lysed by lysing agents that are present in the reagents. For example, the reagents can include a detergent such as NP-40 and/or a protease. The detergent and/or the protease can lyse the cell membrane. In some embodiments, cell lysis may also, or instead, rely on techniques that do not involve a lysing agent in the reagent. For example, lysis may be achieved by mechanical techniques that may employ various geometric features to effect piercing, shearing, abrading, etc. of cells. Other types of mechanical breakage such as acoustic techniques may also be used. Further, thermal energy can also be used to lyse cells. Any convenient means of effecting cell lysis may be employed in the methods described herein.

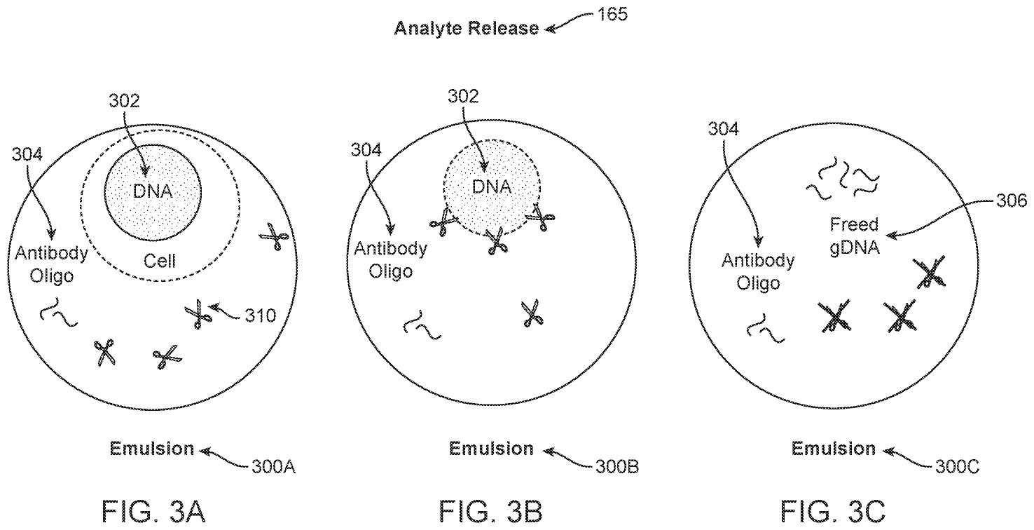

Reference is now made to FIGS. 3A-3C, which depict steps of releasing and processing analytes within an emulsion (e.g., emulsion 300), in accordance with a first embodiment. FIG. 3A depicts emulsion 300A that includes both the cell 102 and reagents 120 (as shown in FIG. 1B). Specifically, in FIG. 3A, the emulsion 300A contains the cell (which further includes DNA 302), antibody oligonucleotides 304 (from the antibodies used to bind cell proteins at step 104 in FIG. 1A), as well as proteases 310 that are added from the reagents. Within the emulsion 300A, the cell is lysed, as indicated by the dotted line of the cell membrane. In one embodiment, the cell is lysed by detergents included in the reagents, such as NP40 (e.g., 0.01% NP40).

FIG. 3B depicts the emulsion 300B as the proteases 302 digest the chromatin-bound DNA 302, thereby releasing genomic DNA. In various embodiments, emulsion 300B is exposed to elevated temperatures to enable the proteases 310 to digest the chromatin. In various embodiments, emulsion 300B is exposed to a temperature between 40° C. and 60° C. In various embodiments, emulsion 300B is exposed to a temperature between 45° C. and 55° C. In various embodiments, emulsion 300B is exposed to a temperature between 48° C. and 52° C. In various embodiments, emulsion 300B is exposed to a temperature of 50° C.

FIG. 3C depicts the free genomic DNA strands 306 and the antibody oligonucleotides 304 residing within emulsion 300C. Proteases 310 are inactivated. In various embodiments, proteases 310 are inactivated by exposing emulsion 300C to an elevated temperature. In various embodiments, emulsion 300C is exposed to a temperature between 70° C. and 90° C. In various embodiments, emulsion 300B is exposed to a temperature between 75° C. and 85° C. In various embodiments, emulsion 300B is exposed to a temperature between 78° C. and 82° C. In various embodiments, emulsion 300B is exposed to a temperature of 80° C.

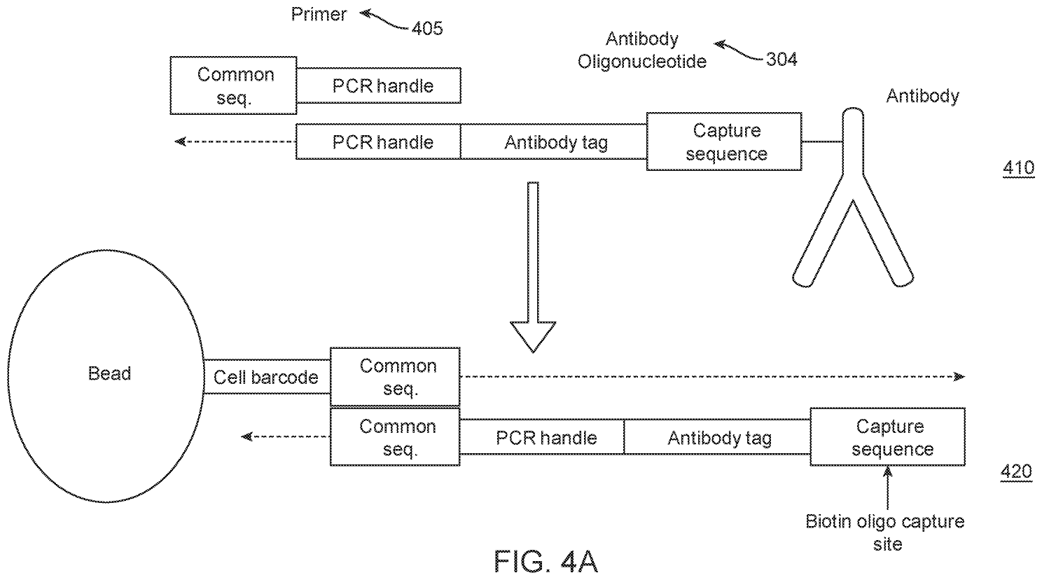

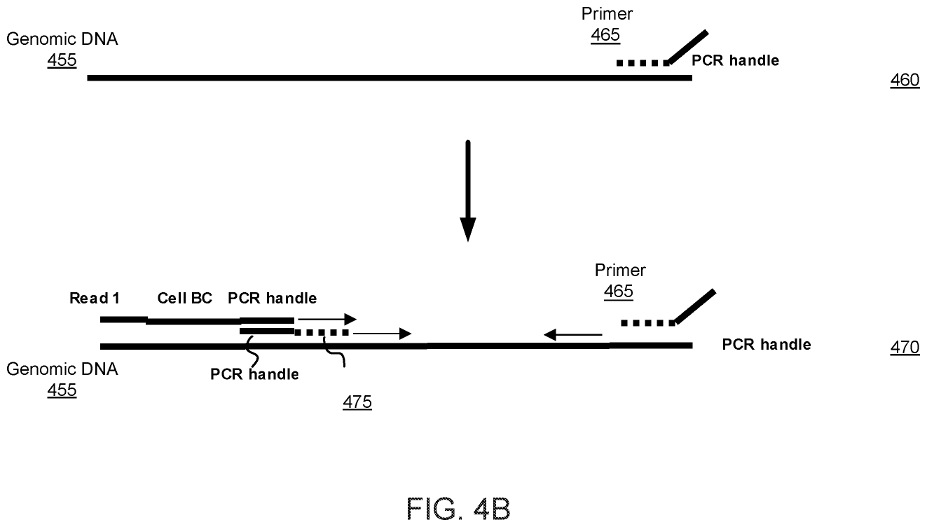

In various embodiments, the antibody oligonucleotide 304 and/or the free genomic DNA 306 undergo priming within emulsion 300C. In various embodiments, reverse primers can hybridize with a portion of the antibody oligonucleotide 304 and/or the free genomic DNA 306. For example, the reverse primer is a gene specific reverse primer that hybridizes with a portion of the free genomic DNA 306. Examples gene specific primers are described in further detail below. As another example, the reverse primer is a PCR handle that hybridizes with a portion of the antibody oligonucleotide 304, which is described in further detail below in relation to FIG. 4A. In various embodiments, the priming of the antibody oligonucleotide 304 can occur earlier, for example in emulsion 300A or emulsion 300B, given that the reverse primers are included in the reagents, which are introduced into emulsion 300A along with the proteases 310.

In various embodiments, the antibody oligonucleotide 304 and the free genomic DNA 306 in emulsion 300C represent at least in part the cell lysate, such as cell lysate 130 shown in FIG. 1B, which is subsequently encapsulated in a second emulsion for barcoding and amplification. Specifically, the step of cell barcoding 170 in FIG. 1 includes encapsulating the cell lysate 130 with a reaction mixture 140 and a barcode 145. In various embodiments, the reaction mixture 140 includes components for performing a nucleic acid reaction on target nucleic acids (e.g., antibody oligonucleotide and freed genomic DNA). For example, the reaction mixture 140 can include primers, enzymes for performing nucleic acid amplification, and dNTPs or ddNTPs for incorporation into amplified nucleic acids.

In various embodiments, a cell lysate is encapsulated with a reaction mixture and a barcode by combining an aqueous phase including the reaction mixture and the barcode with the cell lysate and an immiscible oil phase. In one embodiment, an aqueous phase including the reaction mixture and the barcode are flowed together with a flowing cell lysate and a flowing immiscible oil phase such that water in oil emulsions are formed, where at least one emulsion includes a cell lysate, the reaction mixture, and the barcode. In various embodiments the immiscible oil phase includes a fluorous oil, a fluorous non-ionic surfactant, or both. In various embodiments, emulsions can have an internal volume of about 0.001 to 1000 picoliters or more and can range from 0.1 to 1000 μm in diameter.

In various embodiments, combining the aqueous phase and the immiscible oil phase can be performed in a microfluidic device. For example, the aqueous phase can flow through a microchannel of the microfluidic device to contact the immiscible oil phase, which is simultaneously flowing through a separate microchannel or is held in a stationary reservoir of the microfluidic device. The encapsulated cell lysate, reaction mixture, and barcode within an emulsion can then be flowed through the microfluidic device to perform amplification of target nucleic acids.

Further example embodiments of adding reaction mixture and barcodes to emulsions can include merging emulsions that separately contain the cell lysate and reaction mixture and barcodes or picoinjecting the reaction mixture and/or barcode into an emulsion. Further description of example embodiments of merging emulsions or picoinjecting substances into an emulsion is found in U.S. application Ser. No. 14/420,646, which is hereby incorporated by reference in its entirety.

Once the reaction mixture and barcode are added to an emulsion, the emulsion may be incubated under conditions that facilitate the nucleic acid amplification reaction. In various embodiments, the emulsion may be incubated on the same microfluidic device as was used to add the reaction mixture and/or barcode, or may be incubated on a separate device. In certain embodiments, incubating the emulsion under conditions that facilitates nucleic acid amplification is performed on the same microfluidic device used to encapsulate the cells and lyse the cells. Incubating the emulsions may take a variety of forms. In certain aspects, the emulsions containing the reaction mix, barcode, and cell lysate may be flowed through a channel that incubates the emulsions under conditions effective for nucleic acid amplification. Flowing the microdroplets through a channel may involve a channel that snakes over various temperature zones maintained at temperatures effective for PCR. Such channels may, for example, cycle over two or more temperature zones, wherein at least one zone is maintained at about 65° C. and at least one zone is maintained at about 95° C. As the drops move through such zones, their temperature cycles, as needed for nucleic acid amplification. The number of zones, and the respective temperature of each zone, may be readily determined by those of skill in the art to achieve the desired nucleic acid amplification.

In various embodiments, following nucleic acid amplification, emulsions containing the amplified nucleic acids are collected. In various embodiments, the emulsions are collected in a well, such as a well of a microfluidic device. In various embodiments, the emulsions are collected in a reservoir or a tube, such as an Eppendorf tube. Once collected, the amplified nucleic acids across the different emulsions are pooled. In one embodiment, the emulsions are broken by providing an external stimuli to pool the amplified nucleic acids. In one embodiment, the emulsions naturally aggregate over time given the density differences between the aqueous phase and immiscible oil phase. Thus, the amplified nucleic acids pool in the aqueous phase.

In various embodiments, following pooling, the amplified nucleic acids can undergo further preparation for sequencing. For example, sequencing adapters can be added to the pooled nucleic acids. Example sequencing adapters are P5 and P7 sequencing adapters. The sequencing adapters enable the subsequent sequencing of the nucleic acids.

Example Barcoding of Antibody-Conjugated Oligonucleotide and Genomic DNA

FIG. 4A illustrates the priming and barcoding of an antibody-conjugated oligonucleotide, in accordance with an embodiment. Specifically, FIG. 4A depicts step 410 involving the priming of the antibody oligonucleotide 304 and further depicts step 420 which involves the barcoding and amplification of the antibody oligonucleotide 304. In various embodiments, step 410 occurs within a first emulsion during which cell lysis occurs and step 420 occurs within a second emulsion during which cell barcoding and nucleic acid amplification occurs. In such embodiments, the primer 405 is provided in the reagents and the bead barcode is provided with the reaction mixture. In some embodiments, both steps 410 and 420 occur within the second emulsion. In such embodiments, the primer 405 and the bead barcode shown in FIG. 4A are provided with the reaction mixture.

The antibody oligonucleotide 304 is conjugated to an antibody. In various embodiments, an antibody oligonucleotide 304 includes a PCR handle, a tag sequence (e.g., an antibody tag), and a capture sequence that links the oligonucleotide to the antibody. In various embodiments, the antibody oligonucleotide 304 is conjugated to a region of the antibody, such that the antibody's ability to bind a target epitope is unaffected. For example, the antibody oligonucleotide 304 can be linked to a Fc region of the antibody, thereby leaving the variable regions of the antibody unaffected and available for epitope binding. In various the antibody oligonucleotide 304 can include a unique molecular identifier (UMI). In various embodiments, the UMI can be inserted before or after the antibody tag. In various embodiments, the UMI can flank either end of the antibody tag. In various embodiments, the UMI enables the identification of the particular antibody oligonucleotide 304 and antibody combination.

In various embodiments, the antibody oligonucleotide 304 includes more than one PCR handle. For example, the antibody oligonucleotide 304 can include two PCR handles, one on each end of the antibody oligonucleotide 304. In various embodiments, one of the PCR handles of the antibody oligonucleotide 304 is conjugated to the antibody. Here, forward and reverse primers can be provided that hybridize with the two PCR handles, thereby enabling amplification of the antibody oligonucleotide 304.

Generally, the antibody tag of the antibody oligonucleotide 304 enables the subsequent identification of the antibody (and corresponding protein). For example, the antibody tag can serve as an identifier e.g., a barcode for identifying the type of protein for which the antibody binds to. In various embodiments, antibodies that bind to the same target are each linked to the same antibody tag. For example antibodies that bind to the same epitope of a target protein are each linked to the same antibody tag, thereby enabling the subsequent determination of the presence of the target protein. In various embodiments, antibodies that bind different epitopes of the same target protein can be linked to the same antibody tag, thereby enabling the subsequent determination of the presence of the target protein.

In some embodiments, an oligonucleotide sequence is encoded by its nucleobase sequence and thus confers a combinatorial tag space far exceeding what is possible with conventional approaches using fluorescence. For example, a modest tag length of ten bases provides over a million unique sequences, sufficient to label an antibody against every epitope in the human proteome. Indeed, with this approach, the limit to multiplexing is not the availability of unique tag sequences but, rather, that of specific antibodies that can detect the epitopes of interest in a multiplexed reaction.

Step 410 depicts the priming of the antibody oligonucleotide 304 by a primer 405. As shown in FIG. 4, the primer 405 may include a PCR handle and a common sequence. Here, the PCR handle of the primer 405 is complementary to the PCR handle of the antibody oligonucleotide 304. Thus, the primer 405 primes the antibody oligonucleotide 304 given the hybridization of the PCR handles. In various embodiments, extension occurs from the PCR handle of the antibody oligonucleotide 304 (as indicated by the dotted arrow). In various embodiments, extension occurs from the PCR handle of the primer 405, thereby generating a nucleic acid with the antibody tag and capture sequence.

Step 420 depicts the barcoding of the antibody oligonucleotide 304. As shown in FIG. 4, the barcode (e.g., cell barcode) is releasably attached to a bead and is further linked to a common sequence. Here, the common sequence linked to the cell barcode is complementary to the common sequence linked to the PCR handle, antibody tag, and capture sequence. The antibody oligonucleotide is extended to include the common sequence and cell barcode.

In various embodiments, the antibody oligonucleotide is amplified, thereby generating amplicons with the cell barcode, common sequence, PCR handle, antibody tag, and capture sequence. In various embodiments, the capture sequence contains a biotin oligonucleotide capture site, which enables streptavidin bead enrichment prior to library preparation. In various embodiments, the barcoded antibody-oligonucleotides can be enriched by size separation from the amplified genomic DNA targets.

FIG. 4B illustrates the priming and barcoding of genomic DNA 455, in accordance with an embodiment. Specifically, FIG. 4B depicts step 460 involving the priming of the genomic DNA 455 and further depicts step 470 which involves the barcoding and amplification of the genomic DNA 455. In various embodiments, step 460 occurs within a first emulsion during which cell lysis occurs and step 470 occurs within a second emulsion during which cell barcoding and nucleic acid amplification occurs. In such embodiments, the primer 465 is added in the reagents and the barcode and forward primers shown in step 470 are added with the reaction mixture. In some embodiments, step 460 and step 470 both occur within a single emulsion (e.g., a second emulsion) during which cell barcoding and nucleic acid amplification occurs. In such embodiments, the primer 465 shown in step 460 and the barcode and forward primers shown in step 470 are added with the reaction mixture.

At step 460, a primer 465 (as indicated by the dotted line) hybridizes with a portion of the genomic DNA 455. In various embodiments, the primer 465 is a gene specific primer that targets a sequence of a gene of interest. Therefore, the primer 465 hybridizes with a sequence of the genomic DNA 455 corresponding to the gene of interest. In various embodiments the primer 465 further includes a PCR handle or is linked to a PCR handle.

At step 470, a primer 475 (as indicated by the dotted line) hybridizes with a portion of the genomic DNA 455. In various embodiments, the primer 475 includes a PCR handle or is linked to a PCR handle. In various embodiments, the primer 475 is a gene specific primer that targets another sequence of the gene of interest that differs from the sequence targeted by the primer 465. Additionally, a cell barcode (cell BC), which is releasably attached to a bead, is linked to a PCR handle which hybridizes with the PCR handle of the forward primer. Nucleic acid amplification generates amplicons, each of which include the cell barcode, PCR handle, forward primer, the gene sequence of interest the primer 465, and the PCR handle.

Sequencing and Read Alignment

Amplified nucleic acids (e.g., amplicons) are sequenced to obtain sequence reads for generating a sequencing library. Sequence reads can be achieved with commercially available next generation sequencing (NGS) platforms, including platforms that perform any of sequencing by synthesis, sequencing by ligation, pyrosequencing, using reversible terminator chemistry, using phospholinked fluorescent nucleotides, or real-time sequencing. As an example, amplified nucleic acids may be sequenced on an Illumina MiSeq platform.

When pyrosequencing libraries of NGS fragments are cloned in-situ amplified by capture of one matrix molecule using granules coated with oligonucleotides complementary to adapters. Each granule containing a matrix of the same type is placed in a microbubble of the “water in oil” type and the matrix is cloned amplified using a method called emulsion PCR. After amplification, the emulsion is destroyed and the granules are stacked in separate wells of a titration picoplate acting as a flow cell during sequencing reactions. The ordered multiple administration of each of the four dNTP reagents into the flow cell occurs in the presence of sequencing enzymes and a luminescent reporter, such as luciferase. In the case where a suitable dNTP is added to the 3′ end of the sequencing primer, the resulting ATP produces a flash of luminescence within the well, which is recorded using a CCD camera. It is possible to achieve a read length of more than or equal to 400 bases, and it is possible to obtain 10⁶ readings of the sequence, resulting in up to 500 million base pairs (megabytes) of the sequence. Additional details for pyrosequencing are described in Voelkerding et al., Clinical Chem., 55: 641-658, 2009; MacLean et al., Nature Rev. Microbiol., 7: 287-296; U.S. Pat. Nos. 6,210,891; 6,258,568; each of which is hereby incorporated by reference in its entirety.

On the Solexa/Illumina platform, sequencing data is produced in the form of short readings. In this method, fragments of a library of NGS fragments are captured on the surface of a flow cell that is coated with oligonucleotide anchor molecules. An anchor molecule is used as a PCR primer, but due to the length of the matrix and its proximity to other nearby anchor oligonucleotides, elongation by PCR leads to the formation of a “vault” of the molecule with its hybridization with the neighboring anchor oligonucleotide and the formation of a bridging structure on the surface of the flow cell . These DNA loops are denatured and cleaved. Straight chains are then sequenced using reversibly stained terminators. The nucleotides included in the sequence are determined by detecting fluorescence after inclusion, where each fluorescent and blocking agent is removed prior to the next dNTP addition cycle. Additional details for sequencing using the Illumina platform are found in Voelkerding et al., Clinical Chem., 55: 641-658, 2009; MacLean et al., Nature Rev. Microbiol., 7: 287-296; U.S. Pat. Nos. 6,833,246; 7,115,400; 6,969,488; each of which is hereby incorporated by reference in its entirety.

Sequencing of nucleic acid molecules using SOLiD technology includes clonal amplification of the library of NGS fragments using emulsion PCR. After that, the granules containing the matrix are immobilized on the derivatized surface of the glass flow cell and annealed with a primer complementary to the adapter oligonucleotide. However, instead of using the indicated primer for 3′ extension, it is used to obtain a 5′ phosphate group for ligation for test probes containing two probe-specific bases followed by 6 degenerate bases and one of four fluorescent labels. In the SOLiD system, test probes have 16 possible combinations of two bases at the 3′ end of each probe and one of four fluorescent dyes at the 5′ end. The color of the fluorescent dye and, thus, the identity of each probe, corresponds to a certain color space coding scheme. After many cycles of alignment of the probe, ligation of the probe and detection of a fluorescent signal, denaturation followed by a second sequencing cycle using a primer that is shifted by one base compared to the original primer. In this way, the sequence of the matrix can be reconstructed by calculation; matrix bases are checked twice, which leads to increased accuracy. Additional details for sequencing using SOLiD technology are found in Voelkerding et al., Clinical Chem., 55: 641-658, 2009; MacLean et al., Nature Rev. Microbiol., 7: 287-296; U.S. Pat. Nos. 5,912,148; 6,130,073; each of which is incorporated by reference in its entirety.

In particular embodiments, HeliScope from Helicos BioSciences is used. Sequencing is achieved by the addition of polymerase and serial additions of fluorescently-labeled dNTP reagents. Switching on leads to the appearance of a fluorescent signal corresponding to dNTP, and the specified signal is captured by the CCD camera before each dNTP addition cycle. The reading length of the sequence varies from 25-50 nucleotides with a total yield exceeding 1 billion nucleotide pairs per analytical work cycle. Additional details for performing sequencing using HeliScope are found in Voelkerding et al., Clinical Chem., 55: 641-658, 2009; MacLean et al., Nature Rev. Microbiol., 7: 287-296; U.S. Pat. Nos. 7,169,560; 7,282,337; 7,482,120; 7,501,245; 6,818,395; 6,911,345; 7,501,245; each of which is incorporated by reference in its entirety.

In some embodiments, a Roche sequencing system 454 is used. Sequencing 454 involves two steps. In the first step, DNA is cut into fragments of approximately 300-800 base pairs, and these fragments have blunt ends. Oligonucleotide adapters are then ligated to the ends of the fragments. The adapter serves as primers for amplification and sequencing of fragments. Fragments can be attached to DNA-capture beads, for example, streptavidin-coated beads, using, for example, an adapter that contains a 5′-biotin tag. Fragments attached to the granules are amplified by PCR within the droplets of an oil-water emulsion. The result is multiple copies of cloned amplified DNA fragments on each bead. At the second stage, the granules are captured in wells (several picoliters in volume). Pyrosequencing is carried out on each DNA fragment in parallel. Adding one or more nucleotides leads to the generation of a light signal, which is recorded on the CCD camera of the sequencing instrument. The signal intensity is proportional to the number of nucleotides included. Pyrosequencing uses pyrophosphate (PPi), which is released upon the addition of a nucleotide. PPi is converted to ATP using ATP sulfurylase in the presence of adenosine 5′ phosphosulfate. Luciferase uses ATP to convert luciferin to oxyluciferin, and as a result of this reaction, light is generated that is detected and analyzed. Additional details for performing sequencing 454 are found in Margulies et al. (2005) Nature 437: 376-380, which is hereby incorporated by reference in its entirety.

Ion Torrent technology is a DNA sequencing method based on the detection of hydrogen ions that are released during DNA polymerization. The microwell contains a fragment of a library of NGS fragments to be sequenced. Under the microwell layer is the hypersensitive ion sensor ISFET. All layers are contained within a semiconductor CMOS chip, similar to the chip used in the electronics industry. When dNTP is incorporated into a growing complementary chain, a hydrogen ion is released that excites a hypersensitive ion sensor. If homopolymer repeats are present in the sequence of the template, multiple dNTP molecules will be included in one cycle. This results in a corresponding amount of hydrogen atoms being released and in proportion to a higher electrical signal. This technology is different from other sequencing technologies that do not use modified nucleotides or optical devices. Additional details for Ion Torrent Technology is found in Science 327 (5970): 1190 (2010); US Patent Application Publication Nos. 20090026082, 20090127589, 20100301398, 20100197507, 20100188073, and 20100137143, each of which is incorporated by reference in its entirety.

In various embodiments, sequencing reads obtained from the NGS methods can be filtered by quality and grouped by barcode sequence using any algorithms known in the art, e.g., Python script barcodeCleanup.py . In some embodiments, a given sequencing read may be discarded if more than about 20% of its bases have a quality score (Q-score) less than Q20, indicating a base call accuracy of about 99%. In some embodiments, a given sequencing read may be discarded if more than about 5%, about 10%, about 15%, about 20%, about 25%, about 30% have a Q-score less than Q10, Q20, Q30, Q40, Q50, Q60, or more, indicating a base call accuracy of about 90%, about 99%, about 99.9%, about 99.99%, about 99.999%, about 99.9999%, or more, respectively.

In some embodiments, sequencing reads associated with a barcode containing less than 50 reads may be discarded to ensure that all barcode groups, representing single cells, contain a sufficient number of high-quality reads. In some embodiments, all sequencing reads associated with a barcode containing less than 30, less than 40, less than 50, less than 60, less than 70, less than 80, less than 90, less than 100 or more may be discarded to ensure the quality of the barcode groups representing single cells.

In various embodiments, sequence reads with common barcode sequences (e.g., meaning that sequence reads originated from the same cell) may be aligned to a reference genome using known methods in the art to determine alignment position information. For example, sequence reads derived from genomic DNA can be aligned to a range of positions of a reference genome. In various embodiments, sequence reads derived from genomic DNA can align with a range of positions corresponding to a gene of the reference genome. The alignment position information may indicate a beginning position and an end position of a region in the reference genome that corresponds to a beginning nucleotide base and end nucleotide base of a given sequence read. A region in the reference genome may be associated with a target gene or a segment of a gene. Further details for aligning sequence reads to reference sequences is described in U.S. application Ser. No. 16/279,315, which is hereby incorporated by reference in its entirety. In various embodiments, an output file having SAM (sequence alignment map) format or BAM (binary alignment map) format may be generated and output for subsequent analysis, such as for determining cell trajectory.

Cellular Genotype and Phenotype

Sequencing reads of nucleic acids derived from genomic DNA and the antibody oligonucleotide are analyzed to determine cellular phenotypes and cellular genotypes.

In various embodiments, determining a cell genotype refers to determining one or more mutations in the genome of the cell. In particular embodiments, the Tapestri Insights software is implemented to identify the one or more mutations in the genome of the cell. In one embodiment, the one or more mutations include single nucleotide changes (e.g., SNVs) or short sequences of nucleotide changes (e.g., short indels). Here, aligned sequence reads derived from genomic DNA of the cell are analyzed against the reference genome to determine differences between likely nucleotide bases present in the cell mutations corresponding nucleotide bases present in the reference genome. In various embodiments, identifying SNVs and/or short indels can be accomplished by implementing any publicly available SNV caller algorithms including, but not limited to: BWA, NovoAlign, Torrent Mapping Alignment Program (TMAP), VarScan2, qSNP, Shimmer, RADIA, SOAPsnv, VarDict, SNVMix2, SPLINTER, SNVer, OutLyzer, Pisces, ISOWN, SomVarIUS, and SiNVICT.

In one embodiment, the one or more mutations include structural variants such as CNVs and/or mutations that encompass long sequences (e.g., long indels). Here, split-reads and de-novo assembly methods can used to identify CNVs and/or longer indels. In various embodiments, the CNV caller workflow involves one or more of the following steps: binning, GC content correction, mappability correction, removal of outlier bins, removal of outlier cells, segmentation, and calling of absolute numbers. Further details of CNV caller workflows are described in Fan, X. et al, Methods for Copy Number Aberration Detection from Single-cell DNA Sequencing Data, bioRxiv 696179, which is hereby incorporated by reference in its entirety. In various embodiments, identifying CNVs and/or long indels can be accomplished by implementing any publicly available CNV caller including, but not limited to: HMMcopy, SeqSeg, CNV-seq, rSW-seq, FREEC, CNAseg, ReadDepth, CNVator, seqCBS, seqCNA, m-HMM, Ginkgo, nbCNV, AneuFinder, SCNV, and CNV IFTV.

In various embodiments, sequence reads are pre-processed prior to their use in identifying one or more mutations of the cell genome. For example, reads from a cell are normalized by the cell's total read count and grouped by hierarchical clustering based on amplicon read distribution. Amplicon counts from the cell is divided by the median of the corresponding amplicons from a control group (e.g., a control cell cluster with known CNVs). Thus, normalized percentage of sequencing reads were used to calculate CNVs for each gene.

In various embodiments, sequence reads used to determine the cellular genotype can be derived from various regions of a cell genome. These regions of the cell genome include both coding regions and non-coding regions (e.g., introns, regulatory elements, transcription factor binding sites, chromosomal translocation junctions). Therefore, one or more mutations (e.g., SNVs, CNVs, and indels) can be identified in both coding and non-coding regions. The single-cell workflow analysis detailed above that directly determines cellular genotypes from genomic DNA enables the identification of mutations from both coding and non-coding regions, whereas less direct methods (e.g., those that reverse transcribe RNA) only identify mutations from coding regions.

To determine a cell phenotype, sequence reads derived from antibody-conjugated oligonucleotides are analyzed. Specifically, the sequence of the antibody tag of the antibody oligonucleotide is sequenced. The presence of the sequence read indicates that the corresponding antibody (on which the oligonucleotide was conjugated) had previously been bound to an analyte of the cell. In other words, the presence of the sequence read indicates that the cell expressed the target analyte.

In various embodiments, determining a cell phenotype involves quantifying a level of expression of a target analyte. In various embodiments, quantifying a level of expression of a target analyte involves normalizing the sequence reads derived from antibody-conjugated oligonucleotides. In various embodiments, normalizing the sequence reads involves performing a centered log ratio (CLR) transformation. In various embodiments, normalizing the sequence reads involves performing Denoised and Scaled by Background (DSB). Additional description of DSB normalization is found in Mulè, M. et al. “Normalizing and denoising protein expression data from droplet-based single cell profiling.” bioRxiv 2020.02.24.963603, which is hereby incorporated by reference in its entirety.

In various embodiments, a cell phenotype can refer to the cell expression of 1, 2, 3, 4, 5, 6, 7, 8, 9, 10, 11, 12, 13, 14, 15, 16,17, 18, 19, 20, 21, 22, 23, 24, 25, 26, 27, 28, 29, 30 ,31, 32, 33, 34, 35, 36, 37, 38, 39, 40, 41, 42, 43, 44, 45, 46, 47, 48, 49, 50, 100, 500, 1000, 5000, or 10,000 target analytes. Therefore, the single-cell workflow analysis can yield an expression profile for a plurality of target analytes of a cell.

In various embodiments, the genotype and the phenotype of the cell can be used to classify the cell. For example, the cell can be classified within a population of cells that share at least the genotype, share at least the phenotype, or share at least both the genotype and the phenotype of the cell. In various embodiments, the single-cell workflow analysis is conducted on each cell in a population of cells. Therefore, the cell genotype and cell phenotype of each cell in the population can be used to classify each cell to gain an understanding as to the distribution of cells in the population. In various embodiments, the classified cells provide insight as to the subpopulations that are present. In various embodiments, classifying a cell involves comparing the genotype and phenotype of the cell against a library of known cell populations that are characterized by known genotypes and phenotypes. Therefore, if the cell shares a genotype, shares a phenotype, or shares both a genotype and phenotype with a known cell population, the cell can be classified in a category of the known cell population.

To provide an example, the population of cells can be obtained from a subject suspected of having cancer, each cell in the population can be analyzed using the single-cell workflow to determine each cell's genotype and phenotype. Cells are classified according to their genotypes and phenotypes by comparing to genotypes and phenotypes of known reference cells. Thus, classifying cells in the population using their genotypes and phenotypes reveals a distribution of cells which can guide the selection of a cancer treatment for the subject. For example, if a large proportion of cells in the population are classified with a known cell population that are known to be resistant to particular therapies, then alternative therapies that are more likely to be efficacious can be selected for treating the cancer.

In various embodiments, the genotype and the phenotype of the cell are used to identify subpopulations within a population of cells. This is useful for discovering new subpopulations that were not previously known. For example, a cell population previously thought be homogeneous can be analyzed to reveal multiple subpopulations of cells with different genotype and phenotype combinations. In various embodiments, a cell population may reveal two, three, four, five, six, seven, eight, nine, ten, eleven, twelve, thirteen, fourteen, fifteen, sixteen, seventeen, eighteen, nineteen, or twenty different subpopulations.

In various embodiments, the single-cell workflow analysis is conducted on each cell in a population of cells and the cell genotypes and cell phenotypes of cells in the population are used to identify subpopulations of cells that are characterized by genotypes and phenotypes. In one embodiment, using the genotypes and phenotypes of the cells to identify subpopulations involves performing a dimensionality reduction analysis. In one embodiment, using the genotypes and phenotypes of the cells to identify subpopulations involves performing an unsupervised clustering analysis. In one embodiment, using the genotypes and phenotypes of the cells to identify subpopulations involves performing a dimensionality reduction analysis and an unsupervised clustering analysis.

Examples of unsupervised cluster analysis include hierarchical clustering, k-means clustering, clustering using mixture models, density based spatial clustering of applications with noise (DBSCAN), ordering points to identify the clustering structure (OPTICS), or combinations thereof. Examples of dimensionality reduction analysis include principal component analysis (PCA), kernel PCA, graph-based kernel PCA, linear discriminant analysis, generalized discriminant analysis, autoencoder, non-negative matrix factorization, T-distributed stochastic neighbor embedding (t-SNE), or uniform manifold approximation and projection (UMAP) and dens-UMAP.

In particular embodiments, a dimensionality reduction analysis and unsupervised clustering is performed on at least one of either cellular genotypes or cellular phenotypes of cells in the population. Thus, clusters of cells are generated according to at least one of either cellular genotypes or cellular phenotypes of the cells. In particular embodiments, clusters of cells are generated according to detected SNVs for one or more genes. In particular embodiments, clusters of cells are generated according to detected SNVs for two, three, four, five, six, seven, eight, nine, ten, eleven, twelve, thirteen, fourteen, fifteen, sixteen, seventeen, eighteen, nineteen, twenty, twenty five, thirty, forty, fifty, sixty, seventy, eighty, ninety, or one hundred genes. In particular embodiments, clusters of cells are generated according to detected CNVs for one or more genes. In particular embodiments, clusters of cells are generated according to detected CNVs for two, three, four, five, six, seven, eight, nine, ten, eleven, twelve, thirteen, fourteen, fifteen, sixteen, seventeen, eighteen, nineteen, twenty, twenty five, thirty, forty, fifty, sixty, seventy, eighty, ninety, or one hundred genes. In particular embodiments, clusters of cells are generated according to levels of analyte expression for one or more analytes. In particular embodiments, clusters of cells are generated according to levels of analyte expression for two, three, four, five, six, seven, eight, nine, ten, eleven, twelve, thirteen, fourteen, fifteen, sixteen, seventeen, eighteen, nineteen, twenty, twenty five, thirty, forty, fifty, sixty, seventy, eighty, ninety, or one hundred analytes.

In various embodiments individual cells in clusters are labeled using the other of the cellular genotypes or cellular phenotypes to reveal any subpopulations of cells either within clusters or across the clusters. As one example, cellular phenotypes (e.g., analyte expression) can be used to generate clusters of cells and cellular genotypes (e.g., mutations) are used to label cells in the clusters. As another example, cellular genotypes are used to generate clusters of cells and cellular phenotypes are used to label cells in the clusters.

To provide a specific example, a dimensionality reduction analysis and unsupervised clustering is performed on cellular phenotypes of cells. Specifically, dimensionality reduction analysis can be performed on normalized sequence read values (e.g., CLR values) derived from antibody oligonucleotides. Then, unsupervised clustering is performed on the CLR normalized sequence read values in the dimensionally reduced space to generate clusters of cells. Here, cells that have similar analyte expression profiles may be clustered in a common cluster whereas cells that have dissimilar analyte expression profiles may be clustered in different clusters. Cellular genotypes of the cells can be used to label individual cells within clusters. For example, individual cells within clusters can be labeled as having a particular mutation (e.g., a particular SNV on a gene or an increase/decrease in copy number for a particular gene). In some scenarios, individual cells within clusters can be labeled as having more than one mutation (e.g., SNVs on one or more genes or increase/decrease in copy number of one or more genes).

As another example, a dimensionality reduction analysis and unsupervised clustering is performed on cellular genotypes of cells. Specifically, dimensionality reduction analysis can be performed according to mutations (e.g., SNVs and/or CNVs) of one or more genes identified within the cells. Then, unsupervised clustering is performed in the dimensionally reduced space to generate clusters of cells. Here, cells that have similar genotypes (e.g., mutations of one or more genes) may be clustered in a common cluster whereas cells that have dissimilar genotypes may be clustered in different clusters. Cellular phenotypes of the cells can be used to label individual cells within clusters. For example, individual cells within clusters can be labeled as expressing or not expressing a particular analyte. In some scenarios, individual cells within clusters can be labeled as expressing more than one analyte or not expressing more than one analyte.

In various embodiments, a dimensionality reduction analysis and unsupervised clustering is performed on both cellular genotypes and cellular phenotypes of cells. Here, cells that have similar genotypes (e.g., mutations of one or more genes) and phenotypes may be clustered in a common cluster whereas cells that have dissimilar genotypes and phenotypes may be clustered in different clusters.

Analyzing the labeled clusters of cells can, in some scenarios, reveal subpopulations of cells that have particular combinations of genotypes (e.g., mutations) and phenotypes (e.g., analyte expression). In one embodiment, a subpopulation of cells can refer to a cluster of cells that have a common phenotype and common genotype. For example, a subpopulation of cells can refer to a cluster of cells that express an analyte and have a SNV at a particular position of a gene. As another example, a subpopulation of cells can refer to a cluster of cells that do not an analyte and have an increased copy number of a gene. Any combination of cellular phenotype (e.g., expression or lack of expression of an analyte) and cellular genotype (e.g., presence or absence of one or more SNVs or increase/decrease in copy number of a gene) of a cluster of cells can be identified as a subpopulation.

Cells and Cell Populations

Embodiments described herein involve the single-cell analysis of cells. In various embodiments, the cells are healthy cells. In various embodiments, the cells are diseased cells. Examples of diseased cells include cancer cells, such as cells of hematologic malignancies or solid tumors. Examples of hematologic malignancies include, but are not limited to, acute lymphoblastic leukemia, acute myeloid leukemia, chronic lymphocytic leukemia, chronic myeloid leukemia, classic Hodgkin's Lymphoma, diffuse large B-cell lymphoma, follicular lymphoma, mantle cell lymphoma, multiple myeloma, myelodysplastic syndromes, myeloid, myeloproliferative neoplasms, or T-cell lymphoma. Examples of solid tumors include, but are not limited to, breast invasive carcinoma, colon adenocarcinoma, glioblastoma multiforme, kidney renal clear cell carcinoma, liver hepatocellular carcinoma, lung adenocarcinoma, lung squamous cell carcinoma, ovarian cancer, pancreatic adenocarcinoma, prostate adenocarcinoma, or skin cutaneous melanoma.

In various embodiments, the single-cell analysis is performed on a population of cells. The population of cells can be a heterogeneous population of cells. In one embodiment, the population of cells can include both cancerous and non-cancerous cells. In one embodiment, the population of cells can include cancerous cells that are heterogenous amongst themselves. In various embodiments, the population of cells can be obtained from a subject. For example, a sample is taken from a subject, and the population of cells in the sample are isolated for performing single-cell analysis.

Targeted Panels

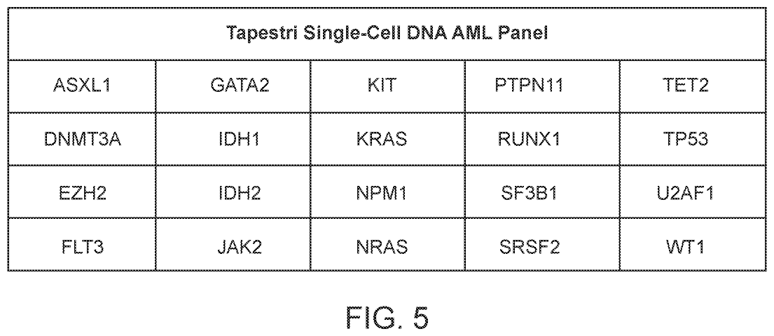

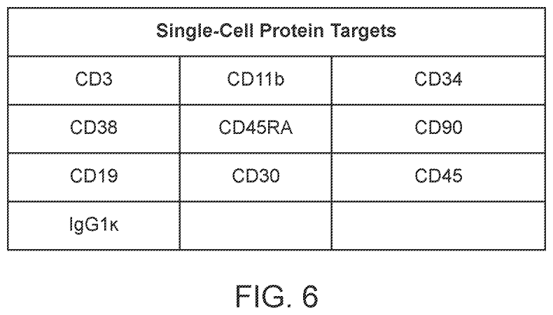

Embodiments disclosed herein include targeted DNA panels for interrogating one or more genes as well as protein panels for interrogating expression and/or expression levels of one or more proteins. In various embodiments, the targeted DNA panels and the protein panels are constructed for particular cancers (e.g., hematologic malignancies and/or solid tumors). FIGS. 5 and 6 show example gene targets and protein targets analyzed using the single cell workflow, in accordance with an embodiment. Specifically, the genes identified in FIG. 5A and the proteins identified in FIG. 5B may be target genes and proteins for a single-cell workflow for detecting or analyzing acute myeloid leukemia.

In various embodiments, the targeted gene panel includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 125, 150, 175, 200, 250, 300, 350, 400, 450, 500, or 1000 genes. In various embodiments, the targeted protein panel includes at least 1, at least 2, at least 5, at least 10, at least 20, at least 30, at least 40, at least 50, at least 60, at least 70, at least 80, at least 90, at least 100, at least 200, at least 300, at least 400, at least 500, or at least 1000 genes.

In various embodiments, the targeted gene panel is specific for detecting cancer and includes one or more genes of ABL1, ADO, AKT1, ALK, APC, AR, ATM, BRAF, CDH1, CDK4, CDKN2A, CSF1R, CTNNB1, DDR2, EGFR, ERBB2, ERBB3, ERBB4, ESR1, EZH2, FBXW7, FGFR1, FGFR2, FGFR3, FLT3, GNA11, GNAQ, GNAS, HNF1A, HRAS, IDH1, IDH2, JAK1, JAK2, JAK3, KDR, KIT, KRAS, MAP2K1, MAP2K2, MET, MLH1, MPL, MTOR, NOTCH1, NRAS, PDGFRA, PIK3CA, PTEN, PTPN11, RAF1, RB1, RET, SMAD4, SMARCB1, SMO, SRC, STK11, TP53, and VHL.

In various embodiments, the targeted gene panel is specific for detecting or analyzing acute lymphoblastic leukemia and includes one or more genes of GNB1, DNMT3A, FAT1, MYB, PAXS, CHD4, ORAI1, TP53BP1, IKZF3, WTIP, BCOR, RPL22, ASXL2, ATRX, IKZF1, KLF9, ETV6, FLT3, HCN4, STAT5B, CNOT3, USP9X, SLC25A33, ZFP36L2, DNAH5, EGFR, ABL1, CDKN1B, FREM2, IDH2, TSPYL2, ASXL1, DDX3X, TAL1, ZEB2, IL7R, BRAF, NOTCH1, KRAS, RB1, CREBBP, MED12, ZNF217, KDM6A, JAK1, IDH1, PIK3R1, EZH2, GATA3, HDAC7, MDGA2, USP7, ZFR2, ITSN1, BCORL1, RPL5, SETD2, EBF1, KMT2C, PTEN, KMT2D, SERPINA1, CTCF, DNM2, RUNX1, PHF6, OVGP1, TBL1XR1, LRFN2, ZFHX4, SORCS1, BTG1, BCL11B, TP53, SMARCA4, ERG, RPL10, NRAS, PIK3CA, CCND3, MYC, WT1, SH2B3, AKT1, NCOR1, EPOR, XBP1, USH2A, LEF1, OPN5, JAK2, LMO2, PTPN11, MGA, NF1, JAK3, SLC5A1, MYCN, FBXW7, PHIP, CDKN2A, CBL, NOS1, SPTBN5, SUZ12, UBA2, and EP300.

In various embodiments, the targeted gene panel is specific for detecting or analyzing chronic lymphocytic leukemia and includes one or more genes of ATM, CHD2, FBXW7, NOTCH1, SPEN, BCOR, CREBBP, KRAS, NRAS, TP53, BIRC3, CXCR4, LRP1B, PLCG2, XPO1, BRAF, DDX3X, MAP2K1, POT1, ZMYM3, BTK, EGR2, MED12, RPS15, CARD11, EZH2, MYD88, SETD2, CD79B, FAT1, NFKBIE, and SF3B1.

In various embodiments, the targeted gene panel is specific for detecting or analyzing chronic myeloid leukemia and includes one or more genes of DNMT3A, CDKN2A, TP53, U2AF1, KIT, ABL1, SETBP1, TET2, ETV6, ASXL1, EZH2, FLT3, and RUNX1.

In various embodiments, the targeted gene panel is specific for detecting or analyzing Classic Hodgkin's Lymphoma and includes one or more genes of B2M, NFKBIA, SOCS1, TNFAIP3, MYB, PRDM1, STAT3, TP53, MYC, REL, and STAT6.

In various embodiments, the targeted gene panel is specific for detecting or analyzing diffuse large B-cell lymphoma and includes one or more genes of ATM, CREBBP, MYD88, STAT6, B2M, EP300, NOTCH1, TET2, BCL2, EZH2, NOTCH2, TNFAIP3, BRAF, FOXO1, PIK3CD, TNFRSF14, CARD11, GNA13, PIM1, TP53, CD79A, CD79B, KMT2D, MYC, PTEN, and SOCS1.

In various embodiments, the targeted gene panel is specific for detecting or analyzing follicular lymphoma and includes one or more genes of TNFRSF14, TNFAIP3, STAT6, CD79B, ARID1A, CARD11, CREBBP, BCL2, NOTCH2, EZH2, SOCS1, EP300, TET2, KMT2D, and TP53.

In various embodiments, the targeted gene panel is specific for detecting or analyzing mantle cell lymphoma and includes one or more genes of ATM, CCND1, NOTCH1, UBR5, BIRC3, KMT2D, TP53, and WHSC1.

In various embodiments, the targeted gene panel is specific for detecting or analyzing multiple myleoma and includes one or more genes of BRAF, FAM46C, IRF4, PIK3CA, CCND1, FGFR3, JAK2, RB1, DIS3, FLT3, KRAS, TP53, DNMT3A, IDH1, NRAS, and TRAF3.

In various embodiments, the targeted gene panel is specific for detecting or analyzing myelodysplastic syndromes and includes one or more genes of ASXL1, FLT3, NF1, TP53, BCOR, GATA2, NRAS, U2AF1, CBL, IDH1, PTPN11, ZRSR2, DNMT3A, IDH2, RUNX1, ETV6, JAK2, SF3B1, EZH2, KRAS, and TET2.

The various embodiments, the targeted gene panel is specific for detecting or analyzing myeloid disease and includes one or more genes of ASXL1, ERG, KDM6A, NRAS, SMC1A, ATM, ETV6, KIT, PHF6, SMC3, BCOR, EZH2, KMT2A, PPM1D, STAG2, BRAF, FLT3, KRAS, PTEN, STAT3, CALR, GATA2, MPL, PTPN11, TET2, CBL, GNAS, MYC, RAD21, TP53, CHEK2, IDH1, MYD88, RUNX1, U2AF1, CSF3R, IDH2, NF1, SETBP1, WT1, DNMT3A, JAK2, NPM1, SF3B1, and ZRSR2.

In various embodiments, the targeted gene panel is specific for detecting or analyzing myeloproliferative neoplasms and includes one or more genes of CSF3R, IDH1, JAK2, ARAF, CHEK2, MPL, KIT, CBL, SETBP1, SF3B1, NRAS, TET2, IDH2, ASXL1, CALR, DNMT3A, EZH2, TP53, RUNX1, NF1, ERBB4, PTPN11, KRAS, and U2AF1.

In various embodiments, the targeted gene panel is specific for detecting or analyzing T-cell lymphoma and includes one or more genes of ALK, CDKN2A, IDH2, RHOA, ARID1A, DDX3X, JAK3, STAT3, ATM, DNMT3A, KMT2C, TET2, CARD11, FAS PLCG1, and TP53.

In various embodiments, the targeted protein panel includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 125, 150, 175, 200, 250, 300, 350, 400, 450, 500, or 1000 proteins. In various embodiments, the targeted protein panel includes at least 1, at least 2, at least 5, at least 10, at least 20, at least 30, at least 40, at least 50, at least 60, at least 70, at least 80, at least 90, at least 100, at least 200, at least 300, at least 400, at least 500, or at least 1000 proteins. In various embodiments, the targeted protein panel includes one or more proteins of HLA-DR, CD10, CD117, CD11b, CD123, CD13, CD138, CD14, CD141, CD15, CD16, CD163, CD19, CD193 (CCR3), CD1c, CD2, CD203c, CD209, CD22, CD25, CD3, CD30, CD303, CD304, CD33, CD34, CD4, CD42b, CD45RA, CD5, CD56, CD62P (P-Selectin), CD64, CD68, CD69, CD38, CD7, CD71, CD83, CD90 (Thy1), Fc epsilon RI alpha, Siglec-8, CD235a, CD49d, CD45, CD8, CD45RO, mouse IgG1, kappa, mouse IgG2a, kappa, mouse IgG2b, kappa, CD103, CD62L, CD11c, CD44, CD27, CD81, CD319 (SLAMF7), CD269 (BCMA), CD99, CD164, KCNJ3, CXCR4 (CD184), CD109, CD53, CD74, HLA-DR, DP, DQ, HLA-A, B, C, ROR1, Annexin A1, or CD20.

Barcodes and Barcoded Beads

Embodiments of the invention involve providing one or more barcode sequences for labeling analytes of a single cell during step 170 shown in FIG. 1. The one or more barcode sequences are encapsulated in an emulsion with a cell lysate derived from a single cell. As such, the one or more barcodes label analytes of the cell, thereby enabling the subsequent determination that sequence reads derived from the analytes originated from the same single cell.

In various embodiments, a plurality of barcodes are added to an emulsion with a cell lysate. In various embodiments, the plurality of barcodes added to an emulsion includes at least 10², at least 10³, at least 10⁴, at least 10⁵, at least 10⁵, at least 10⁶, at least 10⁷, or at least 10⁸ barcodes. In various embodiments, the plurality of barcodes added to an emulsion have the same barcode sequence. For example, multiple copies of the same barcode label are added to an emulsion to label multiple analytes derived from the cell lysate, thereby enabling identification of the cell from which an analyte originates from. In various embodiments, the plurality of barcodes added to an emulsion comprise a ‘unique identification sequence’ (UMI). A UMI is a nucleic acid having a sequence which can be used to identify and/or distinguish one or more first molecules to which the UMI is conjugated from one or more second molecules to which a distinct UMI, having a different sequence, is conjugated. UMIs are typically short, e.g., about 5 to 20 bases in length, and may be conjugated to one or more target molecules of interest or amplification products thereof. UMIs may be single or double stranded. In some embodiments, both a barcode sequence and a UMI are incorporated into a barcode. Generally, a UMI is used to distinguish between molecules of a similar type within a population or group, whereas a barcode sequence is used to distinguish between populations or groups of molecules that are derived from different cells. In some embodiments, where both a UMI and a barcode sequence are utilized, the UMI is shorter in sequence length than the barcode sequence. The use of barcodes is further described in U.S. patent application Ser. No. 15/940,850, which is hereby incorporated by reference in its entirety.

In some embodiments, the barcodes are single-stranded barcodes. Single-stranded barcodes can be generated using a number of techniques. For example, they can be generated by obtaining a plurality of DNA barcode molecules in which the sequences of the different molecules are at least partially different. These molecules can then be amplified so as to produce single stranded copies using, for instance, asymmetric PCR. Alternatively, the barcode molecules can be circularized and then subjected to rolling circle amplification. This will yield a product molecule in which the original DNA barcoded is concatenated numerous times as a single long molecule.

In some embodiments, circular barcode DNA containing a barcode sequence flanked by any number of constant sequences can be obtained by circularizing linear DNA. Primers that anneal to any constant sequence can initiate rolling circle amplification by the use of a strand displacing polymerase (such as Phi29 polymerase), generating long linear concatemers of barcode DNA.

In various embodiments, barcodes can be linked to a primer sequence that enables the barcode to label a target nucleic acid. In one embodiment, the barcode is linked to a forward primer sequence. In various embodiments, the forward primer sequence is a gene specific primer that hybridizes with a forward target of a nucleic acid. In various embodiments, the forward primer sequence is a constant region, such as a PCR handle, that hybridizes with a complementary sequence attached to a gene specific primer. The complementary sequence attached to a gene specific primer can be provided in the reaction mixture (e.g., reaction mixture 140 in FIG. 1). Including a constant forward primer sequence on barcodes may be preferable as the barcodes can have the same forward primer and need not be individually designed to be linked to gene specific forward primers.

In various embodiments, barcodes can be releasably attached to a support structure, such as a bead. Therefore, a single bead with multiple copies of barcodes can be partitioned into an emulsion with a cell lysate, thereby enabling labeling of analytes of the cell lysate with the barcodes of the bead. Example beads include solid beads (e.g., silica beads), polymeric beads, or hydrogel beads (e.g., polyacrylamide, agarose, or alginate beads). Beads can be synthesized using a variety of techniques. For example, using a mix-split technique, beads with many copies of the same, random barcode sequence can be synthesized. This can be accomplished by, for example, creating a plurality of beads including sites on which DNA can be synthesized. The beads can be divided into four collections and each mixed with a buffer that will add a base to it, such as an A, T, G, or C. By dividing the population into four subpopulations, each subpopulation can have one of the bases added to its surface. This reaction can be accomplished in such a way that only a single base is added and no further bases are added. The beads from all four subpopulations can be combined and mixed together, and divided into four populations a second time. In this division step, the beads from the previous four populations may be mixed together randomly. They can then be added to the four different solutions, adding another, random base on the surface of each bead. This process can be repeated to generate sequences on the surface of the bead of a length approximately equal to the number of times that the population is split and mixed. If this was done 10 times, for example, the result would be a population of beads in which each bead has many copies of the same random 10-base sequence synthesized on its surface. The sequence on each bead would be determined by the particular sequence of reactors it ended up in through each mix-split cycle. Additional details of example beads and their synthesis is described in International Application No. PCT/US2016/016444, which is hereby incorporated by reference in its entirety.

Reagents

Embodiments described herein include the encapsulation of a cell with reagents within an emulsion. Generally, the reagents interact with the encapsulated cell under conditions in which the cell is lysed, thereby releasing target analytes of the cell. The reagents can further interact with target analytes to prepare for subsequent barcoding and/or amplification.

In various embodiments, the reagents include one or more lysing agents that cause the cell to lyse. Examples of lysing agents include detergents such as Triton X-100, Nonidet P-40 (NP40) as well as cytotoxins. In some embodiments, the reagents include NP40 detergent which is sufficient to disrupt the cell membrane and cause cell lysis, but does not disrupt chromatin-packaged DNA. In various embodiments, the reagents include 0.01%, 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5.0% NP40 (v/v). In various embodiments, the reagents include at least at least 0.01%, at least 0.05%, 0.1%, at least 0.5%, at least 1%, at least 2%, at least 3%, at least 4%, or at least 5% NP40 (v/v).

In various embodiments, the reagents further include proteases that assist in the lysing of the cell and/or accessing of genomic DNA. Examples of proteases include proteinase K, pepsin, protease—subtilisin Carlsberg, protease type X-Bacillus thermoproteolyticus, protease type XIII—Aspergillus Saitoi. In various embodiments, the reagents includes 0.01 mg/mL, 0.05 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 1.5 mg/mL, 2.0 mg/mL, 2.5 mg/mL, 3.0 mg/mL, 3.5 mg/mL, 4.0 mg/mL, 4.5 mg/mL, 5.0 mg/mL, 6.0 mg/mL, 7.0 mg/mL, 8.0 mg/mL, 9.0 mg/mL, or 10.0 mg/mL of proteases. In various embodiments, the reagents include between 0.1 mg/mL and 5 mg/mL of proteases. In various embodiments, the reagents include between 0.5 mg/mL and 2.5 mg/mL of proteases. In various embodiments, the reagents include between 0.75 mg/mL and 1.5 mg/mL of proteases. In various embodiments, the reagents include between 0.9 mg/mL and 1.1 mg/mL of proteases.

In various embodiments, the reagents can further include dNTPs, stabilization agents such as dithothreitol (DTT), and buffer solutions. In various embodiments, the reagents can include primers, such as reverse primers that hybridize with a target analyte (e.g., genomic DNA or an antibody oligonucleotide). In various embodiments, such primers can be gene specific primers. Example primers are described in further detail below.

Reaction Mixture

As described herein, a reaction mixture is provided into an emulsion with a cell lysate (e.g., see cell barcoding step 170 in FIG. 1). Generally, the reaction mixture includes reactants sufficient for performing a reaction, such as nucleic acid amplification, on analytes of the cell lysate.

In various embodiments, the reaction mixture includes primers that are capable of acting as a point of initiation of synthesis along a complementary strand when placed under conditions in which synthesis of a primer extension product which is complementary to a nucleic acid strand is catalyzed. In various embodiments, the reaction mixture includes the four different deoxyribonucleoside triphosphates (adenosine, guanine, cytosine, and thymine). In various embodiments, the reaction mixture includes enzymes for nucleic acid amplification. Examples of enzymes for nucleic acid amplification include DNA polymerase, thermostable polymerases for thermal cycled amplification, or polymerases for multiple-displacement amplification for isothermal amplification. Other, less common forms of amplification may also be applied, such as amplification using DNA-dependent RNA polymerases to create multiple copies of RNA from the original DNA target which themselves can be converted back into DNA, resulting in, in essence, amplification of the target. Living organisms can also be used to amplify the target by, for example, transforming the targets into the organism which can then be allowed or induced to copy the targets with or without replication of the organisms.

In various embodiments, the contents of the reaction mixture are in a suitable buffer (“buffer” includes substituents which are cofactors, or which affect pH, ionic strength, etc.), and at a suitable temperature.

The extent of nucleic amplification can be controlled by modulating the concentration of the reactants in the reaction mixture. In some instances, this is useful for fine tuning of the reactions in which the amplified products are used.

Primers

Embodiments of the invention described herein use primers to conduct the single-cell analysis. For example, primers are implemented during the workflow process shown in FIG. 1. Primers can be used to prime (e.g., hybridize) with specific sequences of nucleic acids of interest, such that the nucleic acids of interest can be barcoded and/or amplified. Specifically, primers hybridize to a target sequence and act as a substrate for enzymes (e.g., polymerases) that catalyze nucleic acid synthesis off a template strand to which the primer has hybridized. As described hereafter, primers can be provided in the workflow process shown in FIG. 1 in various steps. Referring again to FIG. 1, in various embodiments, primers can be included in the reagents 120 that are encapsulated with the cell 102. In various embodiments, primers can be included in the reaction mixture 140 that is encapsulated with the cell lysate 130. In various embodiments, primers can be included in or linked with a barcode 145 that is encapsulated with the cell lysate 130. Further description and examples of primers that are used in a single-cell analysis workflow process is described in U.S. application Ser. No. 16/749,731, which is hereby incorporated by reference in its entirety.

In various embodiments, the number of distinct primers in any of the reagents, the reaction mixture, or with barcodes may range from about 1 to about 500 or more, e.g., about 2 to 100 primers, about 2 to 10 primers, about 10 to 20 primers, about 20 to 30 primers, about 30 to 40 primers, about 40 to 50 primers, about 50 to 60 primers, about 60 to 70 primers, about 70 to 80 primers, about 80 to 90 primers, about 90 to 100 primers, about 100 to 150 primers, about 150 to 200 primers, about 200 to 250 primers, about 250 to 300 primers, about 300 to 350 primers, about 350 to 400 primers, about 400 to 450 primers, about 450 to 500 primers, or about 500 primers or more.

For targeted DNA sequencing primers in the reagents (e.g., reagents 120 in FIG. 1) may include reverse primers that are complementary to a reverse target sequence on a nucleic acid of interest (e.g., DNA or RNA). In various embodiments, primers in the reagents may be gene-specific primers that target a reverse target sequence of a gene of interest. In various embodiments, primers in the reaction mixture (e.g., reaction mixture 140 in FIG. 1) may include forward primers that are complementary to a forward target sequence on a nucleic acid of interest (e.g., DNA). In various embodiments, primers in the reaction mixture may be gene-specific primers that target a forward target of a gene of interest. In various embodiments, primers of the reagents and primers of the reaction mixture form primer sets (e.g., forward primer and reverse primer) for a region of interest on a nucleic acid. Example gene-specific primers can be primers that target any of the genes identified in the “Targeted Panels” section above.

The number of distinct forward or reverse primers for genes of interest that are added may be from about one to 500, e.g., about 1 to 10 primers, about 10 to 20 primers, about 20 to 30 primers, about 30 to 40 primers, about 40 to 50 primers, about 50 to 60 primers, about 60 to 70 primers, about 70 to 80 primers, about 80 to 90 primers, about 90 to 100 primers, about 100 to 150 primers, about 150 to 200 primers, about 200 to 250 primers, about 250 to 300 primers, about 300 to 350 primers, about 350 to 400 primers, about 400 to 450 primers, about 450 to 500 primers, or about 500 primers or more.

In various embodiments, instead of the primers being included in the reaction mixture (e.g., reaction mixture 140 in FIG. 1) such primers can be included or linked to a barcode (e.g., barcode 145 in FIG. 1). In particular embodiments, the primers are linked to an end of the barcode and therefore, are available to hybridize with target sequences of nucleic acids in the cell lysate.

In various embodiments, primers of the reaction mixture, primers of the reagents, or primers of barcodes may be added to an emulsion in one step, or in more than one step. For instance, the primers may be added in two or more steps, three or more steps, four or more steps, or five or more steps. Regardless of whether the primers are added in one step or in more than one step, they may be added after the addition of a lysing agent, prior to the addition of a lysing agent, or concomitantly with the addition of a lysing agent. When added before or after the addition of a lysing agent, the primers of the reaction mixture may be added in a separate step from the addition of a lysing agent (e.g., as exemplified in the two step workflow process shown in FIG. 1).

A primer set for the amplification of a target nucleic acid typically includes a forward primer and a reverse primer that are complementary to a target nucleic acid or the complement thereof. In some embodiments, amplification can be performed using multiple target-specific primer pairs in a single amplification reaction, wherein each primer pair includes a forward target-specific primer and a reverse target-specific primer, where each includes at least one sequence that is substantially complementary or substantially identical to a corresponding target sequence in the sample, and each primer pair having a different corresponding target sequence. Accordingly, certain methods herein are used to detect or identify multiple target sequences from a single cell sample.

Example System and/or Computer Embodiments

Additionally described herein are systems and computer embodiments for performing the single cell analysis described above. An example system can include a single cell workflow device and a computing device, such as single cell workflow device 106 and computing device 108 shown in FIG. 1A. In various embodiments, the single cell workflow device 106 is configured to perform the steps of cell encapsulation 160, analyte release 165, cell barcoding 170, target amplification 175, nucleic acid pooling 205, and sequencing 210. In various embodiments, the computing device 108 is configured to perform the in silico steps of read alignment 215, determining cellular genotype and phenotype 220, and analyzing cells using cellular genotypes and phenotypes.

In various embodiments, a single cell workflow device 106 includes at least a microfluidic device that is configured to encapsulate cells with reagents, encapsulate cell lysates with reaction mixtures, and perform nucleic acid amplification reactions. For example, the microfluidic device can include one or more fluidic channels that are fluidically connected. Therefore, the combining of an aqueous fluid through a first channel and a carrier fluid through a second channel results in the generation of emulsion droplets. In various embodiments, the fluidic channels of the microfluidic device may have at least one cross-sectional dimension on the order of a millimeter or smaller (e.g., less than or equal to about 1 millimeter). Additional details of microchannel design and dimensions is described in International Patent Application No. PCT/US2016/016444 and U.S. patent application Ser. No. 14/420,646, each of which is hereby incorporated by reference in its entirety. An example of a microfluidic device is the Tapestri™ Platform.

In various embodiments, the single cell workflow device 106 may also include one or more of: (a) a temperature control module for controlling the temperature of one or more portions of the subject devices and/or droplets therein and which is operably connected to the microfluidic device(s), (b) a detection module, i.e., a detector, e.g., an optical imager, operably connected to the microfluidic device(s), (c) an incubator, e.g., a cell incubator, operably connected to the microfluidic device(s), and (d) a sequencer operably connected to the microfluidic device(s). The one or more temperature and/or pressure control modules provide control over the temperature and/or pressure of a carrier fluid in one or more flow channels of a device. As an example, a temperature control module may be one or more thermal cycler that regulates the temperature for performing nucleic acid amplification. The one or more detection modules i.e., a detector, e.g., an optical imager, are configured for detecting the presence of one or more droplets, or one or more characteristics thereof, including their composition. In some embodiments, detector modules are configured to recognize one or more components of one or more droplets, in one or more flow channel. The sequencer is a hardware device configured to perform sequencing, such as next generation sequencing. Examples of sequencers include Illumina sequencers (e.g., MiniSeg™, MiSeg™, NextSeg™ 550 Series, or NextSeg™ 2000), Roche sequencing system 454, and Thermo Fisher Scientific sequencers (e.g., Ion GeneStudio S5 system, Ion Torrent Genexus System).

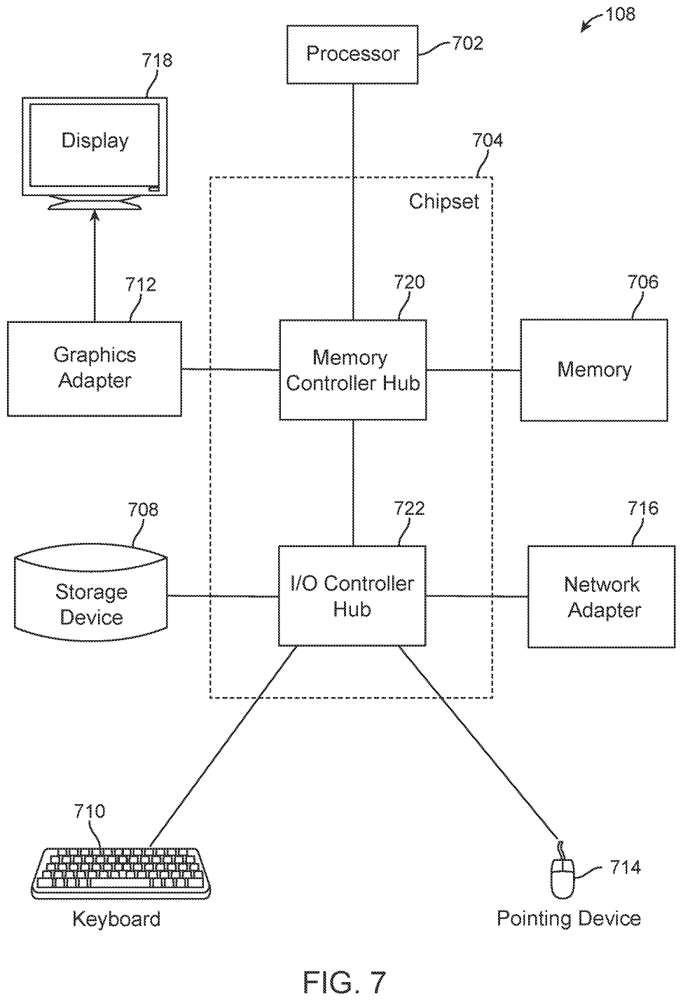

FIG. 7 depicts an example computing device for implementing system and methods described in reference to FIGS. 1-6. For example, the example computing device 108 is configured to perform the in silico steps of read alignment 215 and determining cell trajectory 220. Examples of a computing device can include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, and the like.

FIG. 7 illustrates an example computing device 108 for implementing system and methods described in FIGS. 1-5. In some embodiments, the computing device 108 includes at least one processor 702 coupled to a chipset 704. The chipset 704 includes a memory controller hub 720 and an input/output (I/O) controller hub 722. A memory 706 and a graphics adapter 712 are coupled to the memory controller hub 720, and a display 718 is coupled to the graphics adapter 712. A storage device 708, an input interface 714, and network adapter 716 are coupled to the I/O controller hub 722. Other embodiments of the computing device 108 have different architectures.

The storage device 708 is a non-transitory computer-readable storage medium such as a hard drive, compact disk read-only memory (CD-ROM), DVD, or a solid-state memory device. The memory 706 holds instructions and data used by the processor 702. The input interface 714 is a touch-screen interface, a mouse, track ball, or other type of input interface, a keyboard, or some combination thereof, and is used to input data into the computing device 108. In some embodiments, the computing device 108 may be configured to receive input (e.g., commands) from the input interface 714 via gestures from the user. The graphics adapter 712 displays images and other information on the display 718. For example, the display 718 can show an indication of a predicted cell trajectory. The network adapter 716 couples the computing device 108 to one or more computer networks.

The computing device 108 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 708, loaded into the memory 706, and executed by the processor 702.

The types of computing devices 108 can vary from the embodiments described herein. For example, the computing device 108 can lack some of the components described above, such as graphics adapters 712, input interface 714, and displays 718. In some embodiments, a computing device 108 can include a processor 702 for executing instructions stored on a memory 706.

In various embodiments, methods described herein, such as methods of aligning sequence reads, methods of determining cellular genotypes and phenotypes, and/or methods of analyzing cells using cellular genotypes and phenotypes can be implemented in hardware or software, or a combination of both. In one embodiment, a non-transitory machine-readable storage medium, such as one described above, is provided, the medium comprising a data storage material encoded with machine readable data which, when using a machine programmed with instructions for using said data, is capable of displaying any of the datasets and execution and results of a cell trajectory of this invention. Such data can be used for a variety of purposes, such as patient monitoring, treatment considerations, and the like. Embodiments of the methods described above can be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

Example Kit Embodiments

Also provided herein are kits for performing the single-cell workflow for determining cellular genotypes and phenotypes of populations of cells. The kits may include one or more of the following: fluids for forming emulsions (e.g., carrier phase, aqueous phase), barcoded beads, micro fluidic devices for processing single cells, reagents for lysing cells and releasing cell analytes, reagents and buffers for labeling cells with antibodies, reaction mixtures for performing nucleic acid amplification reactions, and instructions for using any of the kit components according to the methods described herein.

### EXAMPLES

### Example 1: Simultaneous Detection of Cell Surface Protein and Mutations in Single Cells

A mixed population of Jurkat, K562, Mutz-8, and Raji cells were treated with a pool of oligonucleotide-conjugated antibodies that contain 9 monoclonal antibodies of interest plus mouse IgG1k antibody that served as negative control. Cells were then washed and loaded onto the Tapestri Platform to be analyzed with the Single-Cell DNA AML V2 Panel (128 amplicons covering 20 genes). Sequencing data for DNA genotype was processed with the Tapestri Pipeline software and further analyzed with the Tapestri Insights software to determined SNVs.

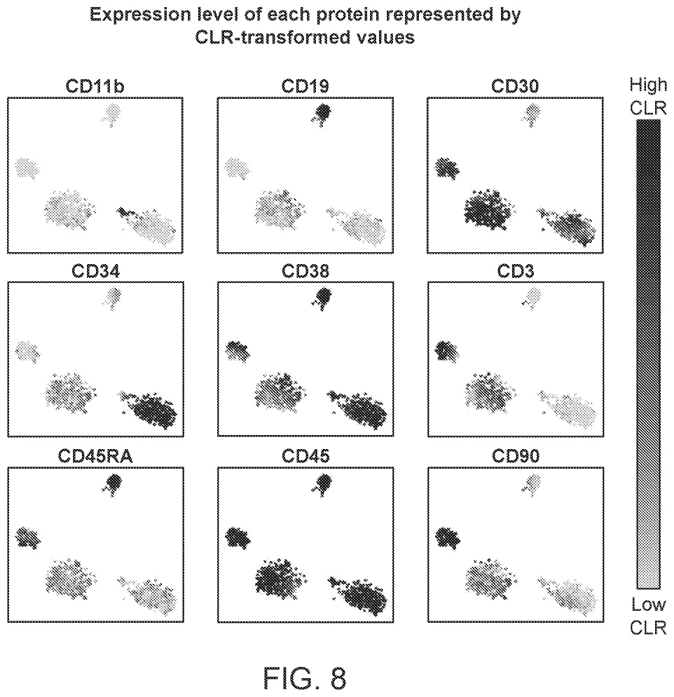

Antibody tag counts were normalized using centered log ratio (CLR) transformation. A t-SNE plot was generated using the CLR values from all protein targets. Specifically, FIG. 8 depicts clustering of cells of a t-SNE plot according to expression of different proteins. As can be seen from FIG. 8, four different clusters of cells with varying protein expressions were identified. Each of the panels reflect CLR values for each respective protein.

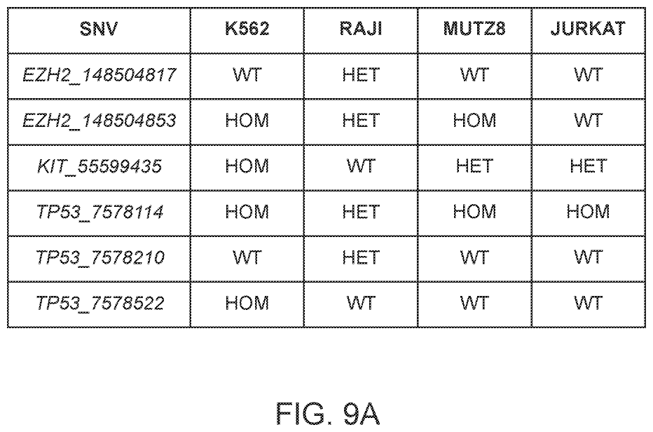

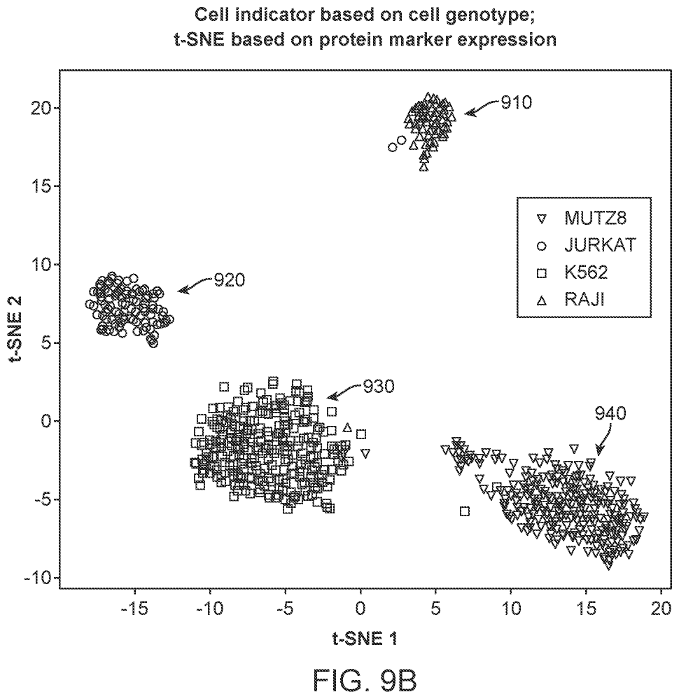

SNV data derived from the cells were analyzed to confirm that the four clusters were the four different cell lines. FIG. 9A depicts four different cell lines and known SNVs that differentiate the cell lines from one another. As such, the SNV data captured from a single cell reveals whether that single cell is a K562 cell, a RAJI cell, MUTZ8 cell, or JURKAT cell.

The SNV data from each cell was next combined with the clustered protein expression data shown in FIG. 8. Specifically, FIG. 9B depicts clustering of cells according to protein expression, with an additional overlay of cell genotype. Specifically, the SNV data reveals that cluster 910 corresponds to RAJI cells, cluster 920 corresponds to JURKAT cells, cluster 930 corresponds to K562 cells, and cluster 940 corresponds to MUTZ8 cells.

Altogether, the single-cell protein marker expression data independently clustered the cells into groups that matched up with the cell genotype data. This demonstrates that the single-cell workflow process is able to successfully classify individual cells into cell populations according to their phenotype (e.g., protein marker expression) and genotype (e.g., SNVs).

### Example 2: CNV Analysis from Targeted DNA Sequencing

CNV data obtained from cells were analyzed to demonstrate that CNV data could be successfully used to differentiate between cells of the four different populations. From the targeted DNA sequencing data, the reads of each cell were first normalized by the cell's total read count and grouped by hierarchical clustering based on amplicon read distribution. A control cell cluster with known CNVs was then identified and amplicon counts from all cells were divided by the median of the corresponding amplicons from the control group. In this experiment, normalized percentage of sequencing reads from the amplicons in the AML panel were used to calculate CNVs for each gene tested. Jurkat cells were used as a control cell line with a known diploid status for all genes tested.

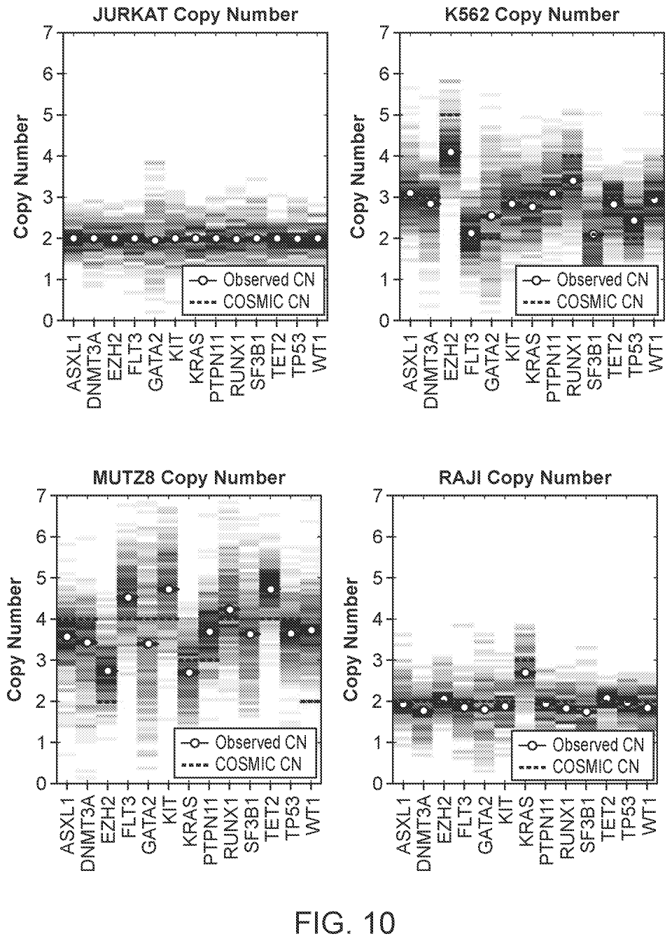

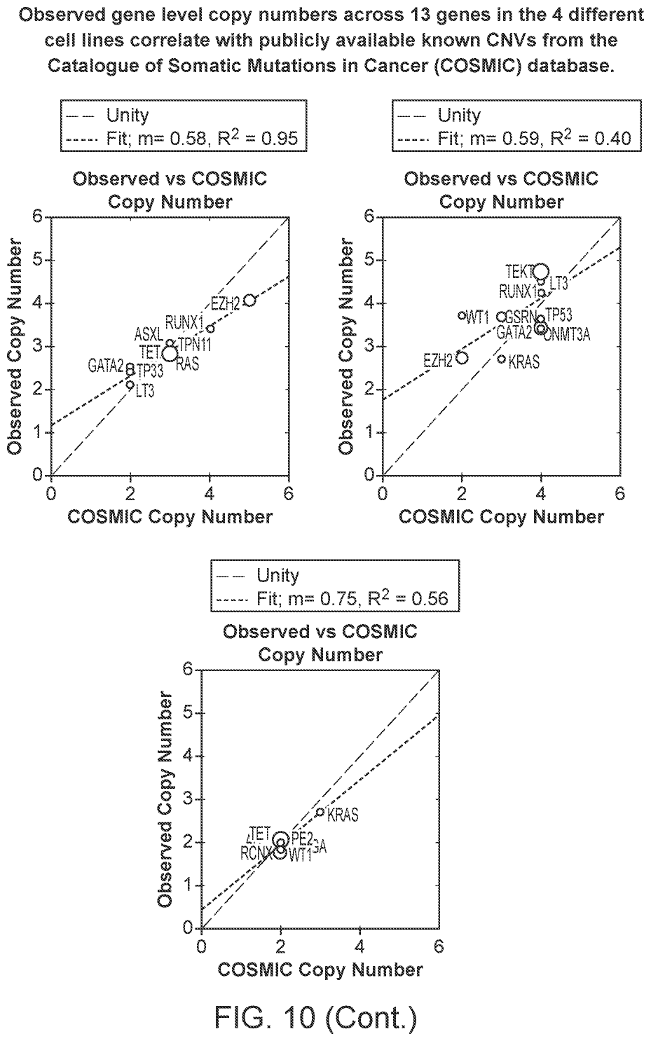

FIG. 10 depicts observed gene level copy numbers for 13 genes across 4 cell lines and the correlation of the observed gene level copy numbers to known levels in the COSMIC database. Generally, FIG. 10 demonstrates that the single-cell workflow process is able to identify a quantity of CNVs for 13 genes across four different cell lines that correlates with publicly available known CNVs (e.g., from COSMIC database).

Specifically, FIG. 10 illustrates the observed copy number and their comparison to the copy numbers in the COSMIC database. As shown in the top row of panels, the observed copy numbers for each of the genes across JURKAT, K562, MUTZ8, and RAJI cells were in agreement with copy numbers in the COSMIC database. As noted before, increased copy numbers of the EZH2 gene is observed in K562 cells, which agrees with the increase copy numbers of the EZH2 gene in the COSMIC database. The same increases were observed in the COSMIC database for the FLT3, KIT, and TET2 genes in MUTZ8 cells, and the KRAS gene in RAJI cells.

The bottom row of panels demonstrate a linear curve fit for the observed copy numbers (y-axis) versus the COSMIC copy number (x-axis). A unity linear fit (slope=1) is shown in each of the panels for comparison purposes.

Altogether, this indicates that the single-cell workflow process successfully identifies copy numbers of genes for individual cells.

### Example 3: Clustering Cell Types by CNV Results

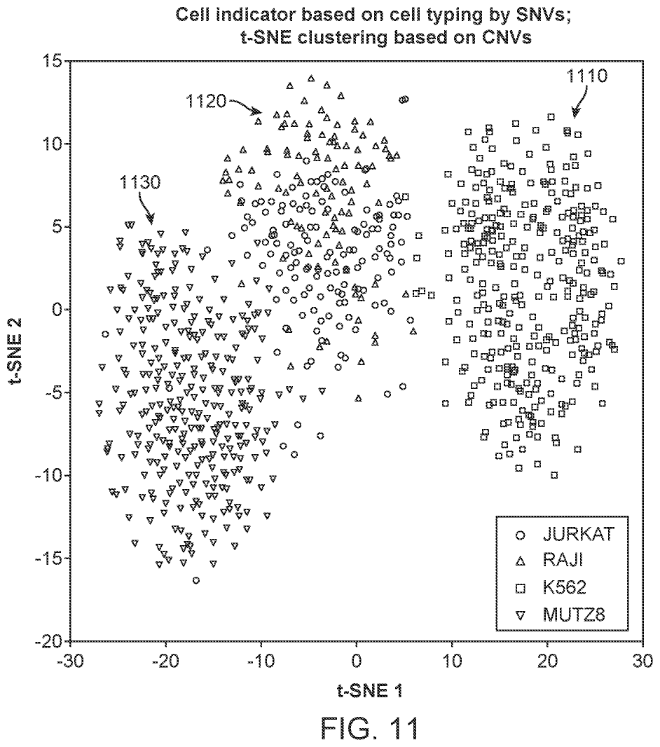

Cells were clustered using t-SNE clustering according to gene CNVs. FIG. 11 depicts clustering of cells according to CNVs with an additional overlay of cell typing by SNVs. Cell typing by SNVs was conducted according to the known SNVs described above in relation to FIG. 9A. Specifically, in FIG. 11, CNV data were grouped on a t-SNE plot and cells different displayed based on SNV genotypes previously established for each cell line.

FIG. 11 shows that the t-SNE clustering according to gene copy numbers resolved three separate clusters 1110, 1120, and 1130. When overlaid with SNV genotyping, the cluster 1110 corresponds to K562 cells, the cluster 1130 corresponds to MUTZ8 cells, and the cluster 1120 corresponds to both JURKAT and RAJI cells. Thus, this demonstrates that the combination of SNV and CNV data enables the classification of cells belonging to different cell types.

### Example 4: Phenotype and Genotype Analysis to Reveal Cell Subpopulations

Raji, K562, TOM1 and KG1 cell lines were analyzed using the Tapestri Single-Cell DNA AML Panel for both SNVs/indels and CNVs. Cells were processed on the Tapestri Platform to simultaneously access protein expression using a panel of 6 antibodies conjugated to analyte barcoded oligo tags. The targets consisted of CD19, CD33, CD45, CD90, HLA-DR and mouse IgG1κ. For downstream analysis, only a select few SNVs/indels, CNVs and proteins were included.

Next, six AML patient samples were analyzed with a custom DNA panel of 31 genes relevant to AML, MPN, and MDS across 109 amplicons. In addition, a custom protein antibody panel was used targeting the following 6 proteins: CD3, CD11b, CD34, CD38, CD45RA and CD90. Data were analyzed with custom Tapestri Pipeline software. SNVs and indels were identified using Tapestri Insights software, CNVs were analyzed using the Mision Bio “tapestri-cnv” package for R, and DNA+protein data were integrated and analyzed using the Mission Bio “tapestri-protein” package for R.

Raji, K562, TOM1 and KG1 cells were mixed together at equal ratios and analyzed for SNVs, indels, CNVs and proteins using the Tapestri Platform.

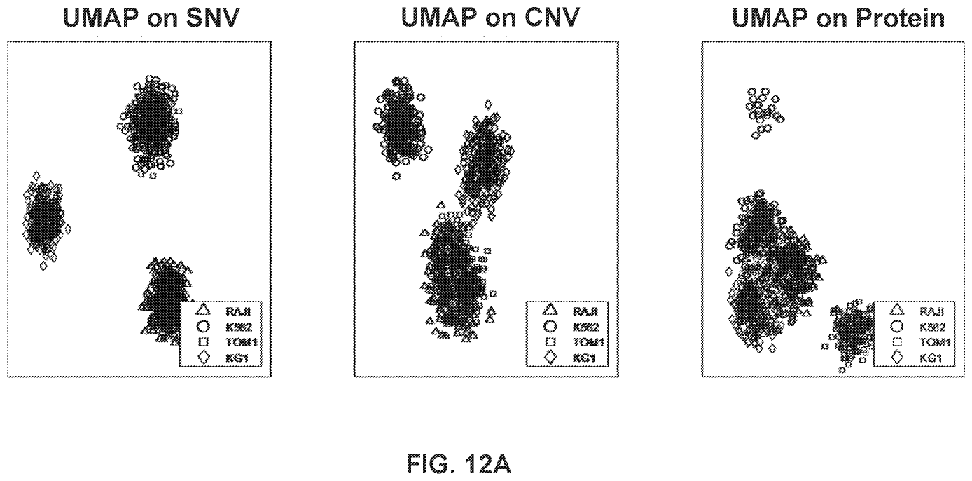

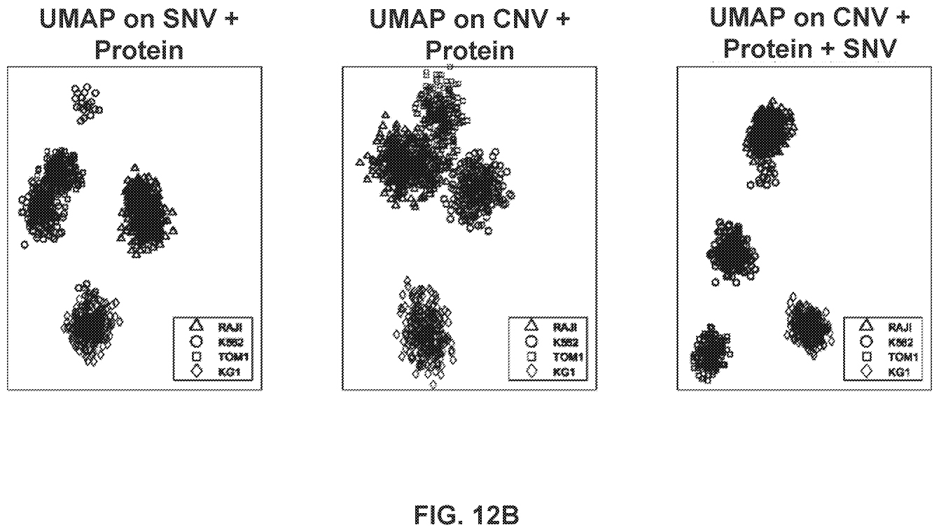

FIG. 12A depicts unsupervised clustering of four cell lines using one of SNV, CNV, and protein expression. Unsupervised clustering (e.g., UMAP) and visualization of each individual analyte resolved 3 cell lines using the SNV data (based on 4 variants). Here, K562 and TOM1 cells were unable to be distinguished while RAJI and KG1 were separately clustered. Unsupervised clustering of CNVs similarly generated 3 clusters with K562 and KG1 cells being separately clustered, but RAJI and TOM1 cells clustered together. Unsupervised clustering of protein expression distinguished the TOM1 cell population, but had overlapping clusters of K562, KG1, and RAJI cell populations.

FIG. 12B depicts unsupervised clustering of four cell lines using at least two of SNV, CNV, and protein expression. Generally, resolution of the cell lines increased when SNV or CNV were combined with protein data respectively, while combined SNV, CNV and protein data together led to the most distinct resolution of the 4 cell line populations. Here, unsupervised clustering using at least two of SNV, CNV, and protein was able to further resolve separate cell populations. Specifically, unsupervised clustering on SNV and protein was able to resolve distinct populations of RAJI cells and KG1 cells, with minimal overlap of K562 and TOM1 cell populations. Similarly, unsupervised clustering of CNV and protein was able to clearly resolve KG1 cells with with minimal overlap between RAJI, TOM1, and K562 cells. Finally, unsupervised clustering of CNV, SNV, and protein fully resolved the four different cell lines. This result illustrates the power of using more data from the same cells with a multi-omics approach to gain the greatest resolution between cell types. This further demonstrates that subpopulations of cells that are mixed in a heterogenous population can be distinguished or identified using the single-cell workflow described herein.

1. A method for analyzing a plurality of cells, the method comprising:
for one or more cells of the plurality of cells:
encapsulating the cell in an emulsion comprising reagents, the cell comprising at least one DNA molecule and at least one analyte-bound antibody conjugated oligonucleotide;
lysing the cell within the emulsion to generate a cell lysate comprising the at least one DNA molecule and the oligonucleotide;
encapsulating the cell lysate comprising the at least one DNA molecule and the oligonucleotide with a reaction mixture in a second emulsion;
performing a nucleic acid amplification reaction within the second emulsion using the reaction mixture to generate amplicons, the amplicons comprising:
a first amplicon derived from one of the at least one DNA molecule; and
a second amplicon derived from the oligonucleotide;
sequencing the first amplicon and the second amplicon;
determining one or more mutations of the cell using at least the sequenced first amplicon;
determining a presence or absence of an analyte using at least the second amplicon; and
discovering a subpopulation of cells in the plurality of cells, the subpopulation of cells characterized by the one or more mutations and the presence or absence of the analyte.

2. The method of claim 1, wherein the one or more mutations comprise a single nucleotide variant (SNV) or a copy number variation (CNV).

3. The method of claim 1, wherein the one or more mutations comprise a single nucleotide variant (SNV) and a copy number variation (CNV).

4. The method of claim 2, wherein discovering the subpopulation of cells in the plurality of cells comprises clustering the one or more cells according to the identified SNV or CNV.

5. The method of claim 4, wherein the SNV or CNV is identified in a gene relevant in acute lymphoblastic leukemia, acute myeloid leukemia, chronic lymphocytic leukemia, chronic myeloid leukemia, classic Hodgkin's Lymphoma, diffuse large B-cell lymphoma, follicular lymphoma, mantle cell lymphoma, multiple myeloma, myelodysplastic syndromes, myeloid, myeloproliferative neoplasms, T-cell lymphoma, breast invasive carcinoma, colon adenocarcinoma, glioblastoma multiforme, kidney renal clear cell carcinoma, liver hepatocellular carcinoma, lung adenocarcinoma, lung squamous cell carcinoma, ovarian cancer, pancreatic adenocarcinoma, prostate adenocarcinoma, or skin cutaneous melanoma.

6. The method of claim 5, wherein the SNV or CNV is identified in any of ABL1, GNB1, KMT2D, PLCG2, GNA13, ATM, BRAF, JAK3, ADO, DNMT3A, SERPINA1, XPO1, PIM1, CCND1, FLT3, STAT3, AKT1, FAT1, CTCF, TP53, NOTCH1, KRAS, ALK, MYB, DNM2, DDX3X, CD79A, UBR5, PTEN, APC, PAX5, RUNX1, MAP2K1, CD79B, BIRC3, KMT2C, AR, CHD4, PHF6, POT1, CALR, TET2, ORAI1, OVGP1, ZMYM3, MYC, GATA2, CARD11, TP53BP1, TBL1XR1, BTK, WHSC1, MPL, FAS, CDH1, IKZF3, LRFN2, EGR2, SOCS1, PTPN11, PLCG1, CDK4, WTIP, ZFHX4, MED12, TNFRSF14, FAM46C, CDKN2A, BCOR, SORCS1, RPS15, TNFAIP3, IRF4, CBL, CSF1R, RPL22, BTG1, STAT6, PIK3CA, GNAS, CTNNB1, ASXL2, BCL11B, EZH2, DDR2, ATRX, MYD88, ARID1A, FGFR3, RAD21, EGFR, IKZF1, SMARCA4, SETD2, JAK2, ERBB2, KLF9, ERG, CREBBP, RB1, CHEK2, ERBB3, ETV6, RPL10, BCL2, DIS3, IDH1, ERBB4, NRAS, NFKBIE, NOTCH2, ESR1, HCN4, SF3B1, STAT5B, CCND3, U2AF1, FBXW7, CNOT3, EP300, CSF3R, FGFR1, USP9X, WT1, IDH2, FGFR2, SLC25A33, SH2B3, NF1, ZFP36L2, KIT, TRAF3, SETBP1, DNAH5, NCOR1, ABL1, ASXL1, GNA11, EPOR, GNAQ, XBP1, CDKN1B, USH2A, NPM1, HNF1A, FREM2, LEF1, HRAS, OPN5, ZRSR2, TSPYL2, LMO2, JAK1, B2M, TAL1, MGA, NFKBIA, ARAF, ZEB2, KDR, IL7R, SLC5A1, MYCN, PRDM1, MAP2K2, PHIP, MET, MLH1, REL, ZNF217, NOS1, MTOR, KDM6A, SPTBN5, SUZ12, UBA2, PDGFRA, PIK3R1, GATA3, CHD2, HDAC7, SMC1A, RAF1, MDGA2, USP7, SPEN, RET, ZFR2, SMAD4, ITSN1, SMARCB1, BCORL1, SMC3, SMO, RPL5, SRC, FOXO1, STK11, EBF1, PIK3CD, KMT2A, RHOA, CXCR4, PPM1D, VHL, LRP1B, and STAG2.

7. The method of claim 1, wherein determining presence or absence of the analyte comprises determining an expression level of the analyte, the analyte bound by the antibody conjugated to the oligonucleotide.

8. The method of claim 7, wherein the analyte is any of HLA-DR, CD10, CD117, CD11b, CD123, CD13, CD138, CD14, CD141, CD15, CD16, CD163, CD19, CD193 (CCR3), CD1c, CD2, CD203c, CD209, CD22, CD25, CD3, CD30, CD303, CD304, CD33, CD34, CD4, CD42b, CD45RA, CD5, CD56, CD62P (P-Selectin), CD64, CD68, CD69, CD38, CD7, CD71, CD83, CD90 (Thy1), Fc epsilon RI alpha, Siglec-8, CD235a, CD49d, CD45, CD8, CD45RO, mouse IgG1, kappa, mouse IgG2a, kappa, mouse IgG2b, kappa, CD103, CD62L, CD11c, CD44, CD27, CD81, CD319 (SLAMF7), CD269 (BCMA), CD99, CD164, KCNJ3, CXCR4 (CD184), CD109, CD53, CD74, HLA-DR, DP, DQ, HLA-A, B, C, ROR1, Annexin A1, or CD20.

9. The method of claim 1, wherein discovering the subpopulation of cells in the plurality of cells comprises clustering the one or more cells according to the determined presence or absence of the analyte.

10. The method of claim 9, wherein clustering the one or more cells according to the identified SNV or CNV or clustering the one or more cells according to the determined presence of the analyte comprises performing a dimensionality reduction analysis selected from any of principal component analysis (PCA), linear discriminant analysis (LDA), T-distributed stochastic neighbor embedding (t-SNE), or uniform manifold approximation and projection (UMAP).

11. The method of claim 1, further comprising:
prior to encapsulating the cell in the emulsion, exposing the cell to a plurality of antibody-conjugated oligonucleotides; and
washing the cell to remove excess antibody conjugated oligonucleotides.

12. The method of claim 11, wherein the oligonucleotides conjugated to the plurality of antibodies comprise a PCR handle, a tag sequence, and a capture sequence.

13. The method of claim 1, wherein the plurality of cells comprise cancer cells.

14. The method of claim 13, wherein the cancer cells are any of acute lymphoblastic leukemia, acute myeloid leukemia, chronic lymphocytic leukemia, chronic myeloid leukemia, classic Hodgkin's Lymphoma, diffuse large B-cell lymphoma, follicular lymphoma, mantle cell lymphoma, multiple myeloma, myelodysplastic syndromes, myeloid, myeloproliferative neoplasms, T-cell lymphoma, breast invasive carcinoma, colon adenocarcinoma, glioblastoma multiforme, kidney renal clear cell carcinoma, liver hepatocellular carcinoma, lung adenocarcinoma, lung squamous cell carcinoma, ovarian cancer, pancreatic adenocarcinoma, prostate adenocarcinoma, or skin cutaneous melanoma.

15. The method of claim 1, further comprising encapsulating a first barcode and a second barcode in the second emulsion along with the at least one DNA molecule, the oligonucleotide, and the reaction mixture.

16. The method of claim 15, wherein the first nucleic acid comprises the first barcode.

17. The method of claim 15, wherein the second nucleic acid comprises the second barcode.

18. The method claim 15, wherein the first barcode and second barcode share a same barcode sequence.

19. The method of claim 15, wherein the first barcode and second barcode share different barcode sequences.

20. The method of claim 15, wherein the first barcode and second barcode are releasably attached to a bead in the second emulsion.
